(19) United States
(12) Patent Application Publication
(10) Pub. No.: US 2016/0030346 A1
(43) Pub. Date: Feb. 4, 2016

(54) ENGINEERED AEROSOL PARTICLES, AND ASSOCIATED METHODS

(71) Applicant: The University of North Carolina at Chapel Hill, Chapel Hill, NC (US)

(72) Inventors: Philip DeSimone, Chapel Hill, NC (US); Benjamin Maynor, Durham, NC (US); Mary Napier, Chapel Hill, NC (US); Jonathan Pillai, Chapel Hill, NC (US); Joseph M. DeSimone, Chapel Hill, NC (US); Will Patrick, Chapel Hill, NC (US); Kyle Laaker, Lexington, KY (US); Hanjun Zhang, Xialu Hebei (CN)

(21) Appl. No.: 14/809,853

(22) Filed: Jul. 27, 2015

Related U.S. Application Data
(63) Continuation of application No. 13/383,518, filed on Jan. 11, 2012, now abandoned.

(51) Int. Cl. A61K 9/16; A61K 9/00

(52) U.S. Cl. CPC A61K 9/16; A61K 9/0073

(57) ABSTRACT

An engineered aerosol particle for use in aerosol applications is provided. The engineered aerosol particle comprises a fabricated nanoparticle body member being non-spherical. The fabricated nanoparticle body member is configured to provide at least one of auto-rotation, tumbling, or lift when entrained in an airstream to thereby increase settling time of the fabricated nanoparticle body member. An associated method is also provided.

### FIELD OF THE INVENTION

Embodiments of the present disclosure relate to engineered particles, and more particularly, to engineered aerosol particles and methods associated therewith.

### BACKGROUND OF THE INVENTION

Particles have been a key component for tens of thousands of products in many different industries. However, up to this point, these particles have, for the most part, been polydisperse in size and shape, with shapes that range from spherical in nature to granulated or globular in shape due to the milled or spray drying processes used to create the particles. In general, particle engineering has not typically included control of size and shape of the engineered particles. Particles for many products, especially for inhaled pharmaceuticals, are intrinsically polydisperse in size and shape due to the milling or spray drying processes used to create the particles. Further, particles have not been designed with rifling or autorotation to generate a leading edge vortex and lift for providing improved aerodynamic characteristics of the particles.

Accordingly, it would be desirable to provide an engineered particle having aerodynamic features/characteristics for providing auto-rotation and/or improved lift when entrained in an airstream so as to provide targeted delivery of the engineered particle to a target site or location. Further, it would be desirable to provide a method for fabricating engineered particles having such aerodynamic features and/or characteristics.

### SUMMARY OF THE INVENTION

Compositions and methods for the design and fabrication of engineered aerosol particles that have utility in multiple fields including delivery of therapeutics to the deep lung and across the blood brain barrier and for use as a novel sensor platform are disclosed. In particular, the particles are capable of autorotation and/or tumbling when entrained in an airstream, to control flight characteristics (akin to rifling) and even to generate lift. These characteristics may also be used to increase settling time of the particles. Such capabilities have never been designed into particle structures before, and it is expected to enable heretofore inaccessible capabilities to address unmet needs.

Embodiments of the present invention include the production of microparticles and/or nanoparticles with predesigned aerodynamic characteristics. Specifically, the particles are designed such that the particles generate auto-rotation, tumbling, and/or lift. The particles can be designed to attain high lift, such as by generating a leading-edge vortex. Likewise particles can be designed for therapeutic delivery via inhalation. Such particles have predetermined shapes to access different regions of the pulmonary system.

Compositions of the present invention include engineered aerosol particles having aerodynamic characteristics. In certain embodiments, the nanoparticles of the present invention can be engineered such that they have precisely controlled particle sizes, shapes, chemical makeups, and/or other particle characteristics. Such precise control over the size and shape of nanoparticles may lead to particles with novel aerodynamic properties. The desired size and shape may depend on the particular application for which a given nanoparticle is intended.

In some aspects, the invention relates to nanoparticles with specific shapes (e.g., asymmetrical or symmetrical shapes) such that the shapes undergo auto-rotation and/or tumbling in an airstream. For example, the particles may be ellipsoid-shaped, Lorenz-shaped, Y-shaped, V-shaped, or L-shaped. In some aspects, the invention relates to nanoparticles designed to create lift, such as through the formation of a leading edge vortex.

In one aspect, the fabricated nanoparticle body member includes at least one fenestration defined completely therethough, wherein the fenestration may be non-circular. The fenestration may also be defined asymmetrically with respect to a central axis of the fabricated nanoparticle body member. Furthermore, the fabricated nanoparticle body member may have an anisotropic density distribution, such as via a particle having plurality of phase-separated materials, porosity, or compositions of different density. In one embodiment, the fabricated nanoparticle body member comprises a particle formed using Particle Replication in Non-wetting Templates.

In some aspects, the particles of the invention may comprise one or more cargos, which endow the particles with various properties. For example, the cargo may be a therapeutic, a targeting agent, an imaging agent, a signaling agent, and/or a sensing agent.

In the therapeutic context, control over the size and shape of particles may enable the particles to be used to access different regions of the pulmonary system upon delivery via inhalation or nasal delivery. In certain embodiments, the sizes of the nanoparticles may be specifically engineered to afford delivery to particular sites within the lung. In certain embodiments, the shapes of the nanoparticles may be engineered such that the particles undergo autorotation and/or tumbling to change the flight characteristics of the particles, opening up opportunities to access various locations within the lung. In some embodiments, multiple sizes and/or shapes of particles may be combined to produce one composition that provides for delivery of particles of different sizes and/or shapes to various sites within the lung. For example, a composition combining particles of different sizes may be designed to deposit certain larger particles in the mouth and the first few generations of airways as well as certain larger particles in the deep lung and the alveolar region.

According to one aspect, an engineered nanoparticle comprises a microfabricated nanoparticle body member being non-spherical and configured to provide at least one of auto-rotation, tumbling, or lift when entrained in an airstream. The nanoparticle body member may also be configured to increase settling time of the fabricated nanoparticle body member. For example, the fabricated nanoparticle body member may settle between about 27-59% slower than equivalent spheres of comparable volume.

Another aspect provides a method of delivering an engineered aerosol nanoparticle. Such a method comprises providing in aerosol form a plurality of nanoparticle body members being non-spherical. Each nanoparticle body member is configured to provide at least one of auto-rotation, tumbling, or lift when entrained in an airstream, which could increase settling time of the fabricated nanoparticle body member. The method further comprises releasing the nanoparticle body members into an airstream.

Still yet another aspect provides a method of fabricating a nanoparticle for use in aerosol applications. The method comprises providing a patterned template and a substrate, wherein the patterned template comprises a patterned template surface having a plurality of recessed areas formed therein. The method further comprises disposing a volume of liquid material in or on the patterned template surface and/or the plurality of recessed areas. The method further comprises forming one or more particles by: (a) contacting the patterned template surface with the substrate and treating the liquid material; and/or (b) treating the liquid material. Each formed particle is non-spherical and is configured to provide at least one of auto-rotation, tumbling, or lift when entrained in an airstream, which could increase settling time of the fabricated nanoparticle body member.

Aspects of the present disclosure thus provide significant advantages as otherwise detailed herein.

### BRIEF DESCRIPTION OF THE DRAWINGS

In order to assist the understanding of embodiments of the invention, reference will now be made to the appended drawings, which are not necessarily drawn to scale. The drawing is exemplary only, and should not be construed as limiting the invention.

FIG. 1 is a schematic view of a system capable of fabricating particles in accordance with various embodiments of the present disclosure;

FIGS. 2A and 2B are scanning electron microscopy (SEM) images and fluorescence microscopy images of shape controlled aerosol particles, according to various embodiments of the present disclosure;

FIGS. 3A-3N illustrate various configurations of engineered particles, according to various aspects of the present disclosure;

FIG. 4 illustrates engineered particles capable of implementation in pulmonary applications, according to one embodiment of the present disclosure;

FIG. 5 illustrates micrographs showing various engineered particles, according to various aspects of the present disclosure;

FIG. 6A-6D illustrates autorotation and leading-edge vortex mechanisms;

FIGS. 7-19 are schematics and micrographs illustrating various aspects of the present disclosure; and

FIGS. 20-26 illustrate various exemplary results from experimental testing according to various aspects of the present disclosure.

### DETAILED DESCRIPTION OF THE PREFERRED EMBODIMENTS

Embodiments of the present inventions now will be described more fully hereinafter with reference to the accompanying drawings. The invention may be embodied in many different forms and should not be construed as limited to the embodiments set forth herein; rather, these embodiments are provided so that this disclosure will satisfy applicable legal requirements. Like numbers refer to like elements throughout.

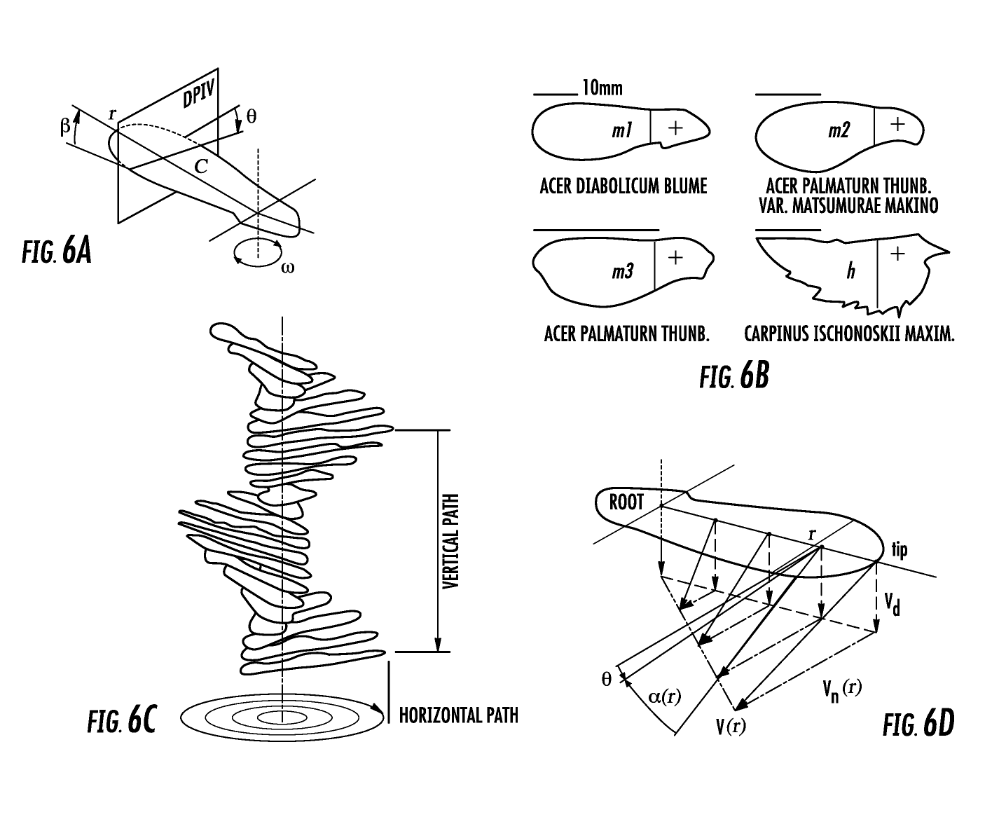

Controlled flight characteristics, including auto-rotation and generation of lift through the creation of a leading edge vortex are of great interest. Autorotation, such as that caused by simple rifling has been shown to fundamentally transform the performance advantages of a bullet over a musket ball. D. Lentink et al. has described the unexpectedly high lift of autorotating seeds of maples and other trees (see FIG. 6), and found that the high lift is attained via a stable leading-edge vortex that develops as the seeds descend (Science 324: 1438-40 (2009)), which is incorporated herein by reference in its entirety. Charles P. Ellington et al. has studied the flight of insects, concluding that the high-lift forces that keep insect flight steady are the result of an intense leading-edge vortex above the wing, which is formed during downstroke movement of the wing (Nature 384: 626-30 (1996)), which is incorporated herein by reference in its entirety.

The development of auto-rotation and the generation of lift through creation of a leading edge vortex have not yet been explored for particles. To date, particles have never been designed with rifling or autorotation to generate a leading edge vortex and lift. Control of the flight performance and characteristics of particles entrained in an air stream could lead to heretofore inaccessible properties with wide utility in a variety of fields.

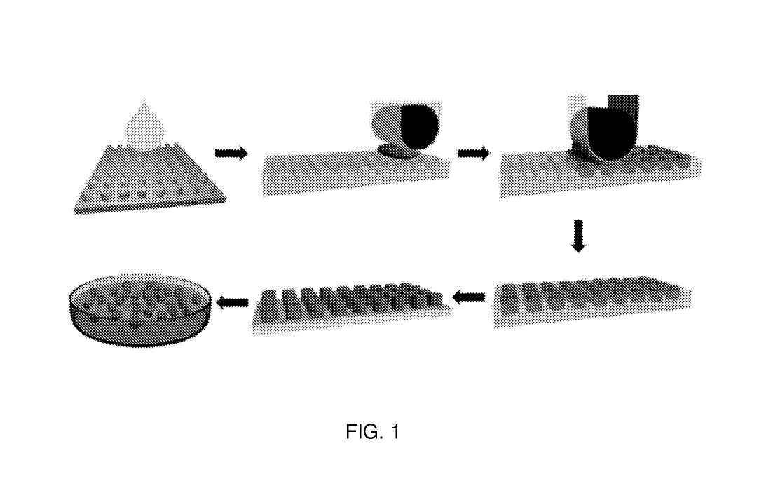

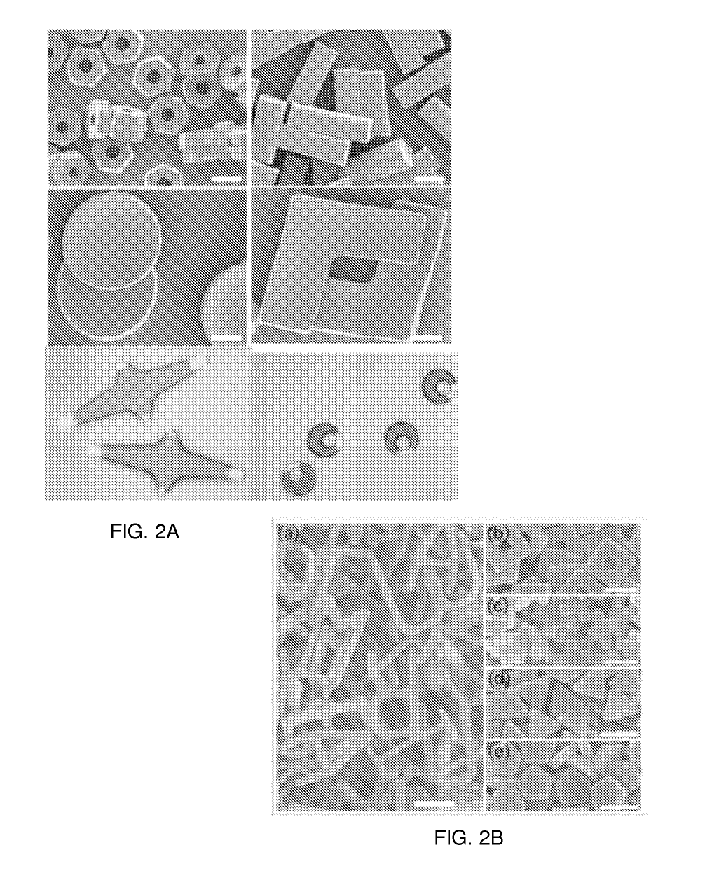

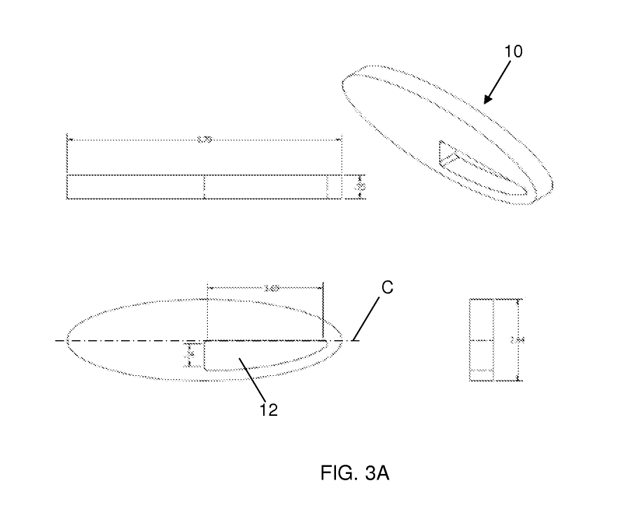

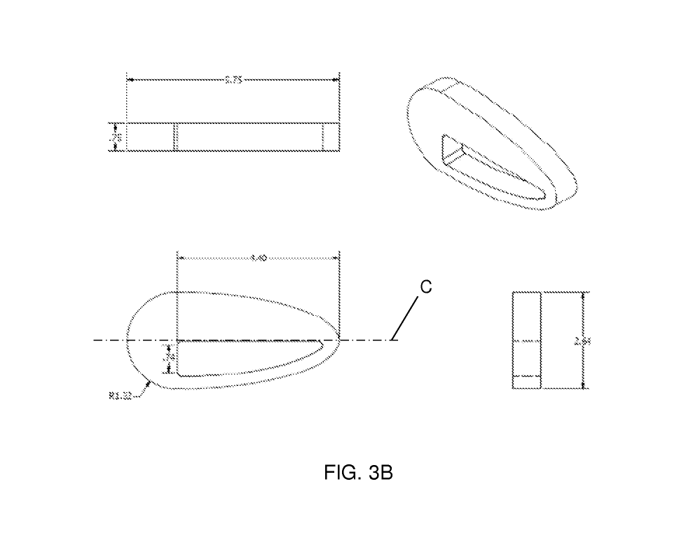

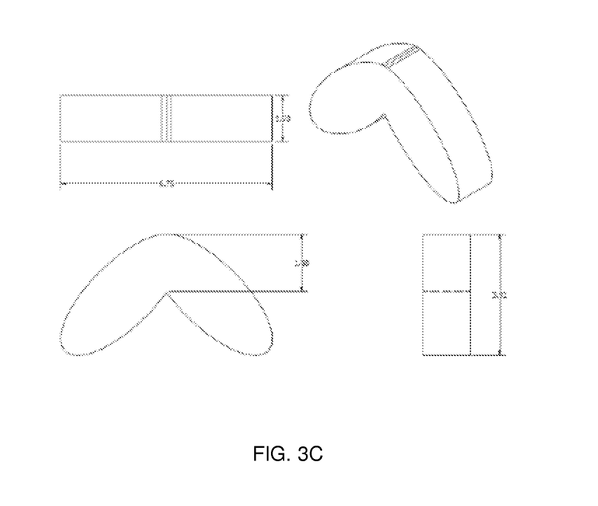

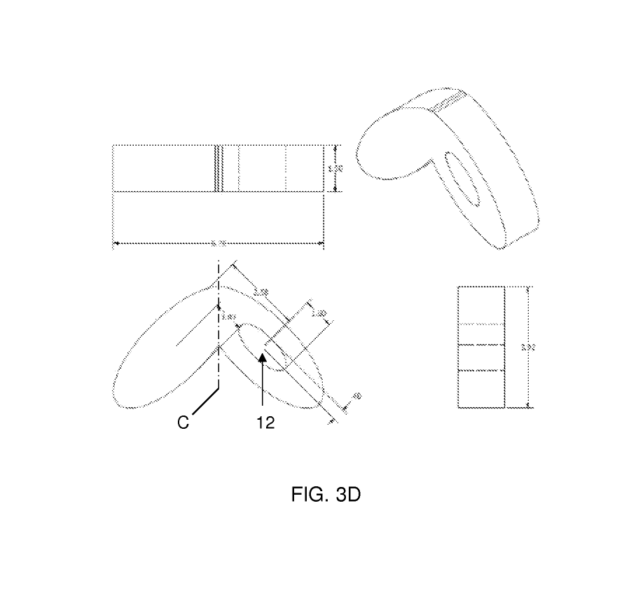

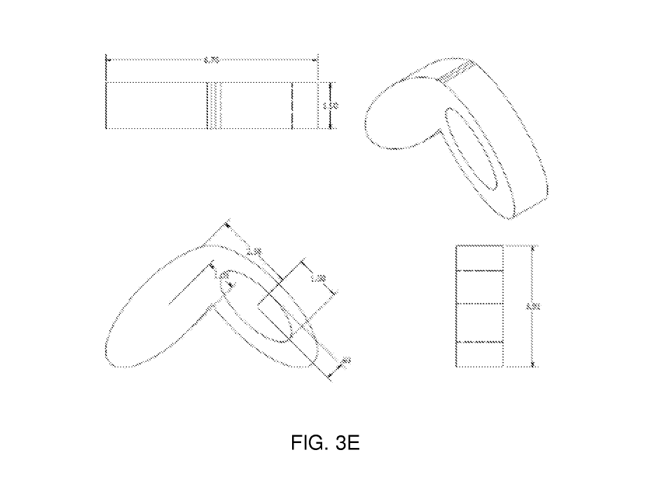

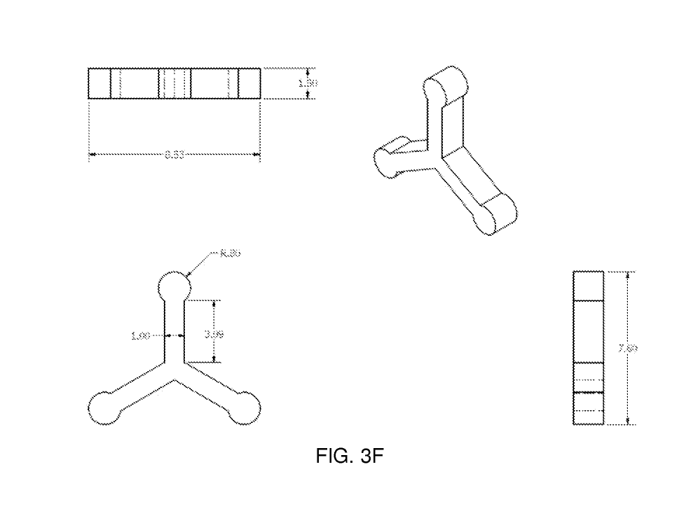

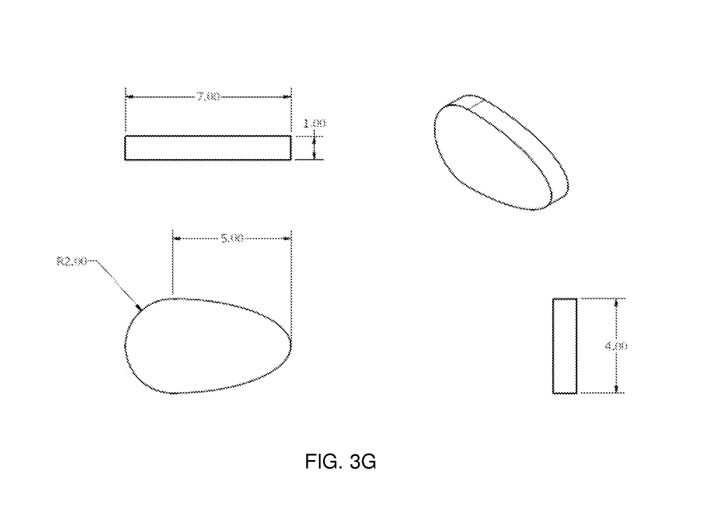

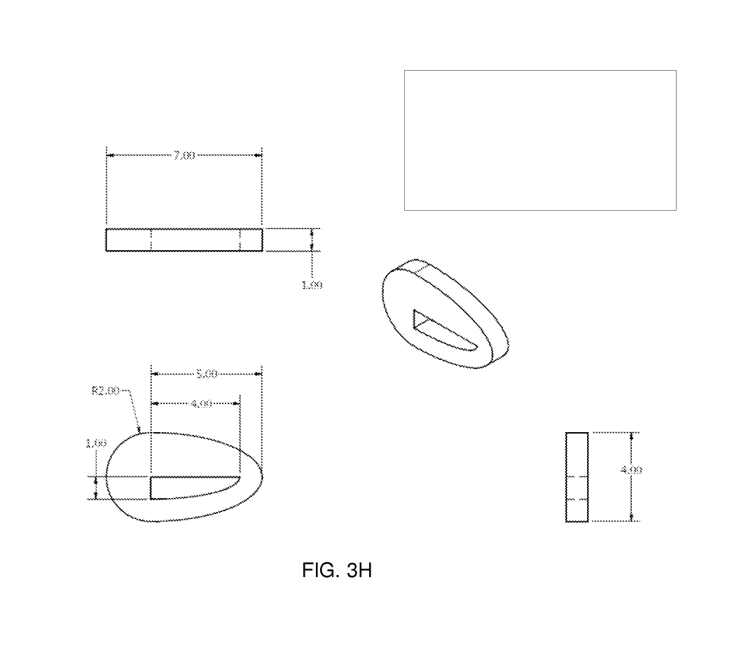

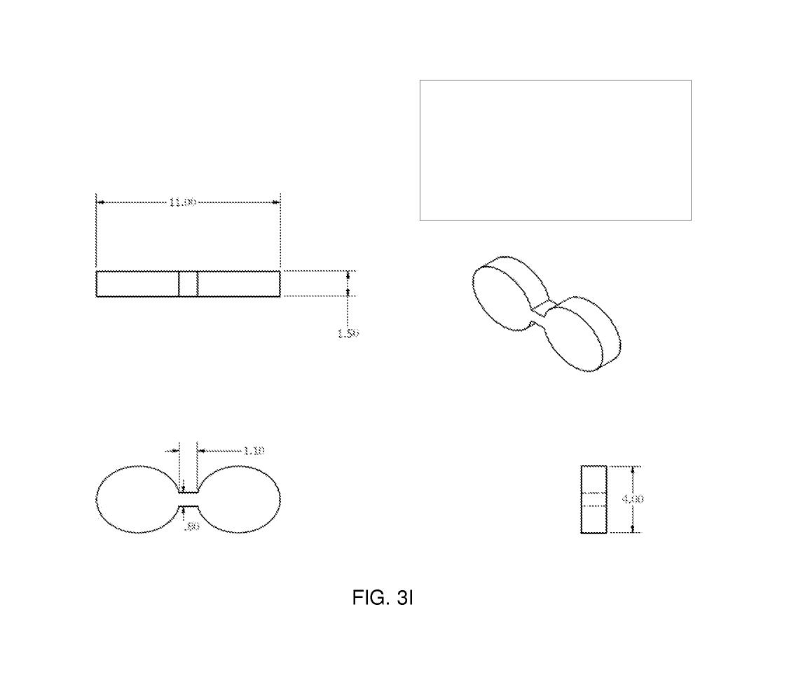

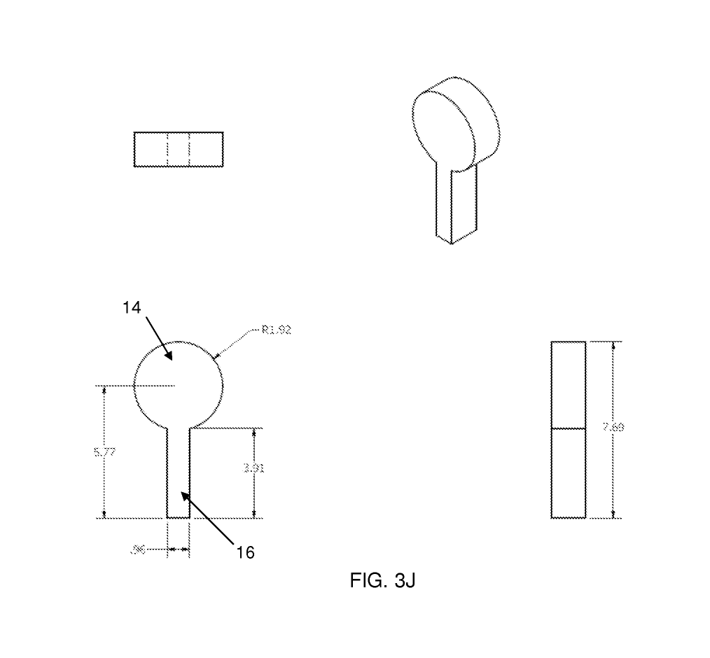

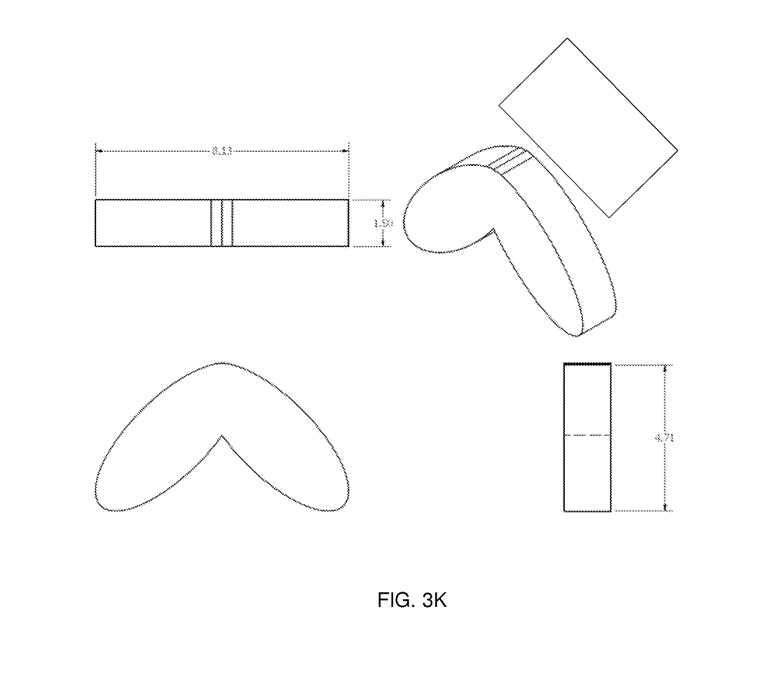

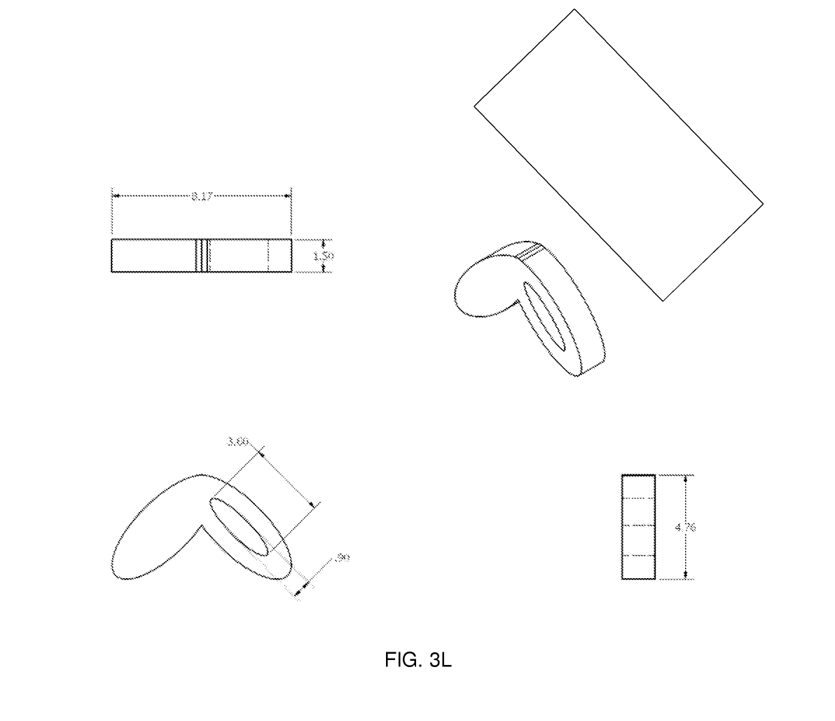

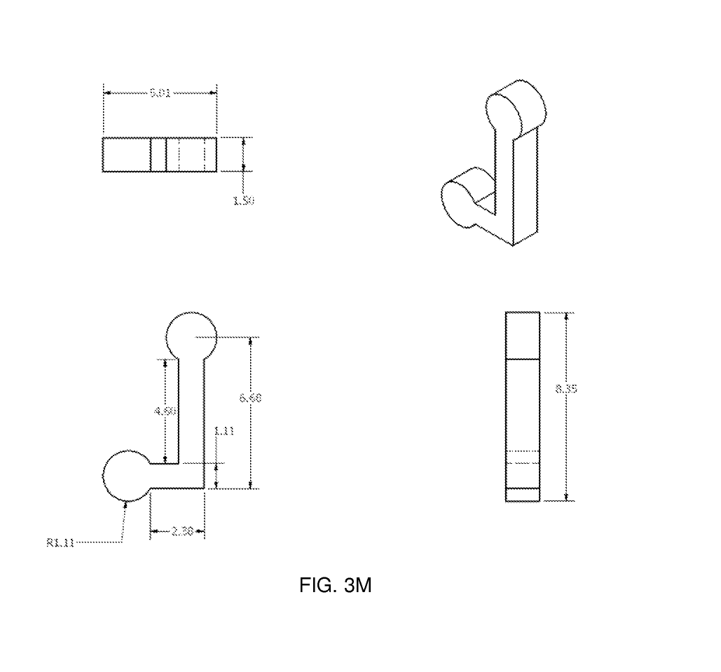

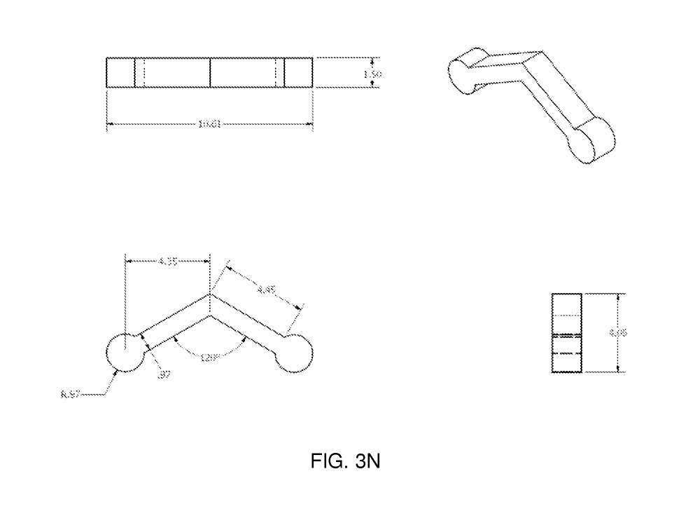

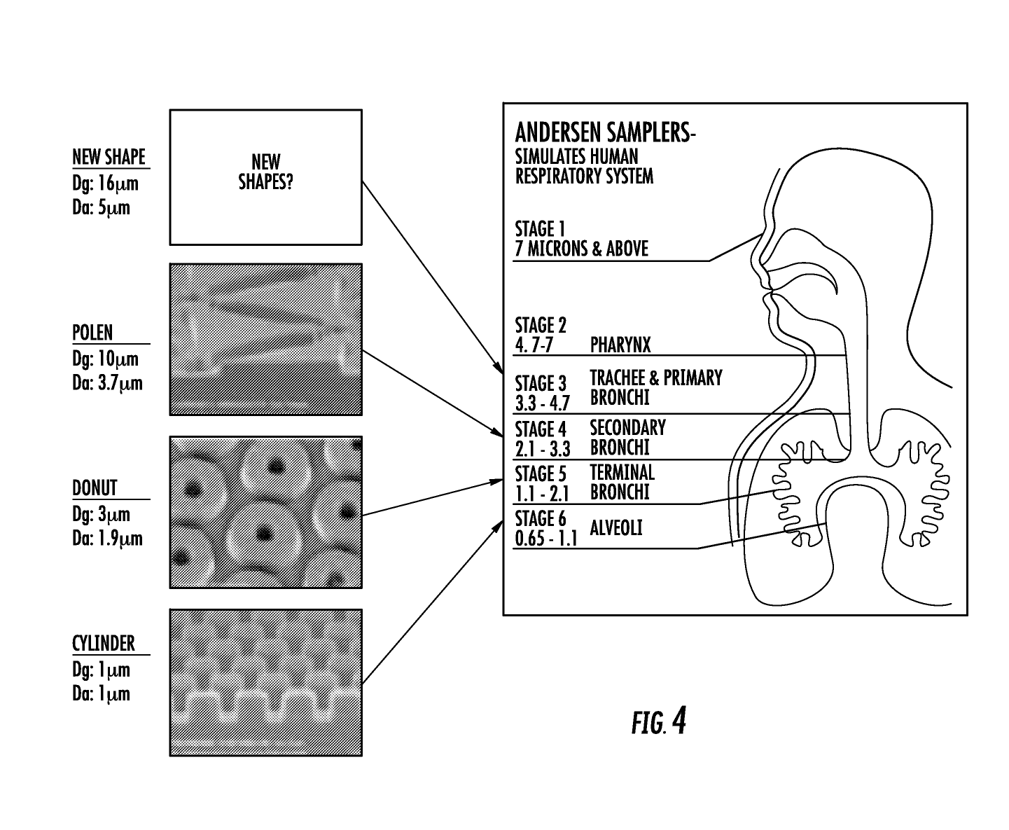

FIGS. 3A-3N illustrate various embodiments of engineered aerosol particles in accordance with the present disclosure. In one embodiment, the fabrication of the particles involves a top-down micro- and nano-fabrication technique PRINT@ (Particle Replication in Non-wetting Templates) (Liquidia Technologies, Inc., Research Triangle Park, N.C.), as generally shown in FIG. 1. See, U.S. Publication No. 2009/0028910 to DeSimone et al., filed Dec. 20, 2004, which is incorporated herein by reference in its entirety. PRINT@ is a platform technology that enables the generation of engineered micro- and nano-particles having precisely controlled size, shape, chemical make-up and functionality. PRINT@ is the first scalable top-down fabrication process useful for making organic and inorganic, shape-controlled, engineered particles and 2-D arrays of particles. PRINT@ is amenable to continuous roll-to-roll fabrication techniques that can enable the scale-up of these new materials to practical levels for the building of various prototype devices. In this regard, unique particle shapes may be designed and fabricated using PRINT, a continuous roll-to-roll nano- and micro-fabrication process. In some instances, the shape-specific engineered aerosols may be comprised of therapeutics, vaccines and chemical/biological sensors.

The engineered aerosol particles disclosed herein may be fabricated using PRINT@ technology, which allows predetermined engineering of the parameters of an ideal nanoparticle delivery vehicle. PRINT@ technology utilizes liquid polymers or Fluorocur™ (Liquidia Technologies, Inc., Research Triangle Park, N.C.) to replicate micro or nano sized structures on a master template. The polymers utilized in PRINT@ molds are liquid at room temperature and can be photo-chemically cross-linked into elastomeric solids that enable high resolution replication of micro or nano sized structures. The liquid polymer is then cured while in contact with the master, thereby forming a replica image of the structures on the master. Upon removal of the cured liquid polymer from the master template, the cured liquid polymer forms a patterned template that includes cavities or recess replicas of the micro or nano-sized features of the master template and the micro or nano-sized cavities in the cured liquid polymer can be used for high-resolution micro or nanoparticle fabrication. For more detailed description of the materials used to fabricate the molds of the present invention and methods of molding micro or nanoparticles in the molds see: U.S. patent application Ser. No. 10/583,570, filed Jun. 19, 2006, Ser. No. 11/594,023 filed Nov. 7, 2006, Ser. No. 11/921,614 filed Jun. 17, 2005, Ser. No. 11/879,746 filed Jul. 17, 2007, Ser. No. 11/633,763 filed Dec. 4, 2006, Ser. No. 12/162,264 filed Jan. 14, 2009, Ser. No. 12/439,281 filed Sep. 30, 2009, Ser. No. 12/250,461 filed Oct. 13, 2008, and Ser. No. 12/630,569 filed Dec. 3, 2009; and PCT International Patent Application Serial Nos.: PCT/US04/42706, filed Dec. 20, 2004; PCT/US/06/23722, filed Jun. 19, 2006; PCT/US06/34997, filed Sep. 7, 2006; PCT/US06/43305, filed Nov. 7, 2006; PCT/US07/02476, filed Jan. 29, 2007, PCT/US09/36068 filed Mar. 4, 2009, PCT/US/09/041559 filed Apr. 23, 2009, and PCT/US09/041652 filed Apr. 24, 2009; each of which is incorporated herein by reference in its entirety. See also, U.S. Provisional Patent Application Ser. No. 60/531,531, filed Dec. 19, 2003; 60/583,170, filed Jun. 25, 2004; 60/604,970 filed Aug. 27, 2004; 60/691,607, filed on Jun. 17, 2005; 60/714,961, filed Sep. 7, 2005; 60/762,802, filed Jan. 27, 2006; 60/798,858, filed May 9, 2006; 60/734,228, filed Nov. 7, 2005; 60/757,411, filed Jan. 9, 2006; 60/799,876, filed May 12, 2006; 60/833,736, filed Jul. 27, 2006; and 60/828,719, filed Oct. 9, 2006; each of which is incorporated herein by reference it its entirety.

As shown in FIG. 1, the master template (grey) is fabricated using advanced lithographic techniques. A unique liquid fluoropolymer (green) is then added to the surface of the master template and photochemically crosslinked (top row, left), then peeled away to generate a precise mold having micro- or nanoscale cavities (upper middle). The low surface energy and high gas permeability of the PRINT® mold enables liquid precursors (red) to particles to fill the cavities (top row, right) through capillary rise. The inter-connecting “flash” layer of liquid wetting the land area between the cavities is not formed (bottom row, right). Once the liquid in the mold cavities is converted to a solid, the array of particles (red) can be removed (bottom row, middle) from the mold (green) by bringing the mold in contact with an adhesive layer (yellow).

According to some embodiments, the methods of the invention are drawn to: i) the development of the PRINT® technique for fabricating engineered aerosol particles with features down to the 100 nm length scale; ii) the evaluation of the aerosolization characteristics of various engineered particle shapes, including computation fluid dynamics analyses; iii) the demonstration of the utility of PRINT® for making engineered aerosol particles that provide new capabilities in the delivery of therapeutics to the lung and to the central nervous system (CNS); and iv) the evaluation of opportunities for incorporating sensing, signaling, and taggant capabilities onto engineered aerosol particles.

The particles of the invention may be fabricated to be specific and designed shapes that lead to auto-rotation, tumbling, and/or lift when the particles are caught in an airstream. These characteristics may be configured to increase settling time of the particle. Accordingly, such particles may be useful as a new sensor platform for the evaluation of aerosol clouds at a distance. In this regard, the engineered sensor platforms may be capable of traveling across the globe much in the way that tons of desert dust moves through the atmosphere each year from the Sahara regions of North Africa across oceanic barriers. In some instances, it is envisioned that auto-rotating particles, when designed like rifling for a bullet, could enable inhaled particles to more easily navigate the pulmonary tree to allow the deposition and delivery of cargos to the deep lung. Such a development could be very impactful for the delivery of vaccines and treatments for bacterial infections, cystic fibrosis, emphysema and lung cancer. The particles may also be engineered to cross the blood brain barrier via intranasal routes. Such particles may be useful for the treatment of pain through the delivery of drugs directly to the central nervous system (CNS) or for the treatment of other brain diseases including Parkinson's disease and brain cancer. Accordingly, engineered aerosols in accordance with the present disclosure may be fabricated and mass produced using a continuous, roll-to-roll process that is able to generate shape-controlled micro- and nano-particles in quantities of sufficient scale to be suitable for deployment in the field. Additionally, such particles may be embodied with attributes that enable function such as, for example, surveillance, chemical/biological detection and mitigation, and therapeutic capabilities.

Particles have been a key component for tens of thousands of products in many different industries. However, up to this point, these particles have, for the most part, been polydisperse in size and shape, with shapes that range from spherical in nature to granulated or globular in shape due to the milled or spray drying processes used to create the particles. Our approach for fabricating particles, referred to as PRINT, is a top-down approach that exploits the micro- and nano-fabrication techniques from the semiconductor industry, extended to a high throughput, continuous roll-to-roll process, to make engineered particles. PRINT® is unique over the imprint lithography techniques promulgated by Whitesides et al⁸ in that PRINT® uses elastomeric fluoropolymers (photochemically curable perfluoropolyethers [PFPEs]) instead of silicones which results in three distinct features not possible with silicones: i) PFPEs have a much lower surface energy⁹⁻¹³ which enables the selective filling of nano-scale cavities in the mold using any organic liquid—without wetting the land area around the cavities—which enables distinct objects or particles to be formed at the micro- and nano-scales without the formation of an interconnecting “flash layer”; ii) organic liquids and sol-gel metal oxide precursors do not swell fluoropolymers like they do silicones; and iii) the Teflon-like characteristics of the PFPE mold allows the resultant particles to be easily harvested from the mold. PRINT® allows the fabrication of precisely defined micro- and nano-particles with control over particle size (20 nm to >20 micron), shape, chemical composition (organic/inorganic, solid/porous), cargo (magnetite, biosensors, therapeutics, proteins, oligonucleotides, siRNA, RFID tags, imaging agents), modulus (stiff, deformable) and surface chemistries (antibodies, PEG chains, metal chelators), including the spatial distribution of ligands on the particle. Our previous studies have shown the ability to make precisely defined PRINT particles from a wide range of chemistries including dozens of different hydrogel materials, biodegradable polylactides, titania, barium titanate, tin oxide, etc. PRINT® is the only particle technology that can create truly engineered particles from such a diverse range of chemistries in form factors that include free-flowing powders, isotropic and external field (electric and magnetic) aligned colloidal dispersions, and 2- and 3-dimensional arrays of nanoparticles.

Shape specific PRINT® particles may be used for the targeted delivery of chemotherapy agents and vaccines via intravenous injections. Size and shape of micro- and nano-particles (made from polymeric hydrogels) plays a role in fundamental biological processes such as endocytosis and biodistribution in whole animals.⁴ Further, PRINT® particles may be used as imaging agents and carriers of low MW cytotoxins and biological cargos like siRNA.² Recently we demonstrated the ability to make particles out of pure biological materials using PRINT®.⁷

Embodiments of the present invention exploit PRINT® to make particles of controlled size and shape in order to engineer aerosol particles. Engineered aerosol particles could have significant potential to address unmet needs in a myriad of applications. Specifically, embodiments of the present disclosure may be used to: i) develop the PRINT® technique for fabricating engineered aerosol particles with features down to the 100 nm length scale; ii) evaluate the aerosolization characteristics using scattering techniques and Anderson Cascade Impactor analyses of various engineered particle shapes, including computation fluid dynamics analyses; iii) demonstrate the utility of PRINT® for making engineered aerosol particles that provide new capabilities in the delivery of therapeutics to the lung and to the CNS, and iv) evaluate the opportunities for incorporating sensing, signaling, and taggant capabilities onto engineered aerosol particles.

1) Design and Fabrication of Engineered Aerosol Particles.

The effective design of an engineered aerosol requires a number of requisite design criteria including a) particle shapes that lead to low packing densities (non-nesting shapes), b) low inter-particle interactions (to prevent particle aggregation and bulk powders with long shelf life), c) appropriate sizes and shapes to yield in-flight characteristics for a given application, and d) particle chemical composition to effect the desired functionality. Understanding and controlling the distribution of inhaled therapeutics is one of the biggest challenges in the field. The traditional view is that particle deposition is governed by three mechanisms: impaction, sedimentation, and diffusion, which are influenced by particle slip, shape and density. Sophisticated aerodynamic physics can predict the flow properties for particles delivered using any number of inhalers and can predict the deposition sites in the lung.¹⁴ Aerodynamic diameter (dₐₑ) is one of the most important parameters in aerodynamic physics and is a strong predictor for how well particles enter the lungs, how far they travel in the lungs and where they will deposit. Large particles (dₐₑ>5 μm) mainly deposit in the mouth and the first few generation of airways by inertial impaction.¹⁵ The deposition of smaller particles (1<dₐₑ<5 μm) is dictated by a combination of inertial impaction and sedimentation and mainly deposit in the central and peripheral airways and in the alveolar lung region. The deposition of very fine particles (dₐₑ<1 μm) is controlled by diffusion and are usually exhaled and cannot deposit efficiently in the lung. Particles with dₐₑ between 1 and 5 μm are usually desired for drug delivery purposes since they can reach the deep lung and the alveolar region.^{16,17} Another emerging strategy is to develop “large porous particles” (LPP). With density less than unity, LPPs can have geometric diameters in the range of 10-20βM.¹⁸ It has been found that LPP particles are easier to disperse from packed dry powder when compared to smaller particles. Furthermore, these large particles are not sequestered by macrophages as easily as the smaller particles and may allow for a more sustained therapeutic effect.

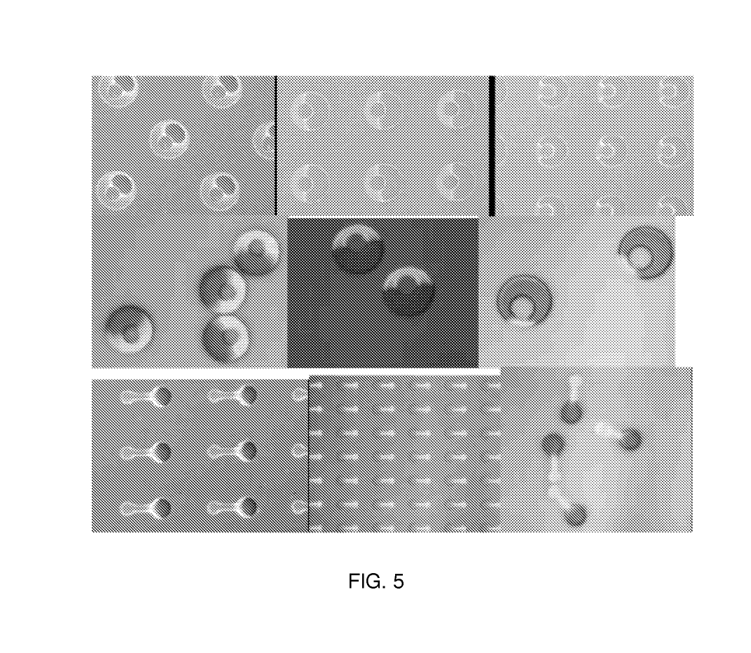

Building on this understanding, embodiments of the present disclosure take into account the role that particle shape also plays on the aerosolization characteristics of particles. In particular it is envisioned that autorotation plays a role in the flight characteristics of particles much in the way that rifling affects the trajectory of bullets versus a musket ball. Heretofore, no one has been able to investigate the role of auto-rotation on the trajectory of particles. However, there are some interesting studies on the role of autorotation in the dispersal characteristics of seeds. Indeed in a recent issue of Science¹⁹, researchers report the achievement of a leading edge vortex of “helicopter” maple seeds which contributes to their dispersability. In accordance with various aspects of the present disclosure, a systematic series of particle shapes, as shown in FIGS. 3A-3N, may be fabricated to explore the role that shape will have on the aerosolization characteristics of particles. Some of the particle designs may induce auto-rotation and/or tumbling when the particles are entrained in an airstream. In addition, some of the shapes may be designed to create lift through the formation of a leading edge vortex. If a thinning of one of the edges of the particle is required to create such a vortex, partial filling of the mold with a dissolvable component may be done to create a thinned edge, as shown in FIG. 5. Surface asperities or controlled surface roughness up to 100 nm in height may also provide a mechanism for lowering inter-particle interactions by creating geometries that are non-nesting to frustrate agglomeration.

With reference to FIGS. 3C, 3D, 3E, 3K, and 3L, such a configuration is generally referred to as a “Lorenz” shape. The Lorenz attractor is a chaotic attractor based on a simplification of the Navier-Stokes Equations used to study convective flow in a given area. These equations are shown below:

dx/dt=σ*(y−x)

dy/dt=ρ*x−y−x*z

dz/dt=x*y−b*z

### Where σ is the Pradlt Number,

$$σ = \frac{Fluid Viscosity}{Thermal Conductivity}$$

ρ is the difference in temperature between the top and bottom of the container
b is the height ratio of the “box” considered.
x,y,z are spatial coordinates.

Because σ, p, and b are user defined parameters, the Lorenz attractors can vary greatly in shape.

Also, the Lorenz attractor is a 3-Dimensional system and most common views are simply projections onto either the xy, yz, or xz planes. As shown, the “Lorenz” shaped particles may have varied lobe and aperture sizes. The actual “Lorenz” particle diagrams are composed of two congruent ellipses joined at a right angle. Although this is not mathematically equivalent to a projection of the Lorenz attractor, the shape it models roughly mimics the projected appearance of the inspiring attractor. Such a configuration may provide asymmetric mass distribution and differences in aerodynamic properties between left and right lobes. The lobe having the aperture may experience different aerodynamic behavior both due to the center of mass being pulled left as well as differing shear and pressure forces from the airflow as the particle falls. Such a shape may induce some form of rotation.

According to some embodiments, the engineered particles have an off-axis center of mass, which may be generated by asymmetric and/or non-spherical shapes (e.g., primarily from 2-D features with uniform thickness). The off-axis center of mass may also be generated by anisotropy in mass or density distribution (e.g., fenestrations or apertures and/or different phase-separated materials). Each particle 10 may include one or more fenestration 12 to create different tumbling characteristics, wherein the fenestration can be any cavity, hole, aperture, or the like that is defined completely or partially through the particle. As shown in FIGS. 3A, 3B, 3D, 3H, and 3L, the fenestration 12 may have an ellipsoid, elongated, or non-circular shape, although other shapes may be used if desired in order to alter the aerodynamic characteristics of the particle. In addition, the fenestration 12 may be defined asymmetrically with respect to a central axis “C” of the particle (see e.g., FIG. 3A). The engineered particles may be further configured to create lift such as via leading-edge vortices. Moreover, the engineered particles may include surface functionalization for stealth and/or targeting functionality. In addition, the engineered particles may have non-interlocking features for ease of aerosolization. Also, the engineered particles may include truly 3-D leading or trailing edges (aerofoil cross-sections and variable thickness features).

As mentioned above, FIGS. 3A-3N illustrate various exemplary particle shapes. In this regard, FIGS. 3A, 3B, 3G, and 3H illustrate ellipsoid-shaped particles, FIGS. 3C, 3D, 3E, 3K, and 3L depict “Lorenz”-shaped particles, and FIGS. 3F, 3I, 3J, 3M and 3N depict “ball-and-stick” configurations. The ball-and-stick shape may be any shape having a one or more rectangular or elongated portions 16 with one or more rounded or spherical portions 14 (see e.g., FIG. 3J). For example, the ball-and-stick configurations could be 1, L, V, Y, X, or “dumbbell”-shaped. The ball-and-stick configurations may also include a multitude of rigid or flexible portions (e.g., string-like arms) joined to a central or hub portion, or elongated portions that are configured to be constrained into a globular or spherical shape and expanded during flight. Moreover, in the instance where the particles include a plurality of ball-and-sticks, the ball-and-sticks may be radially or symmetrically aligned with respect to one another and may be a two-dimensional (e.g., Y-shaped) or three-dimensional (e.g., tripod shaped) particle.

The particle can be designed to influence its aerodynamic flight characteristics. Broadly speaking, the particles are designed to exhibit three primary flight characteristics: autorotation, tumbling, and/or the generation of lift. The primary flight mode is likely to predispose the particle to produce a specific deposition pattern in vivo as elucidated below. Autorotation can be further differentiated into in-plane rotation that is tightly centered about a central axis or spiral motion that is bound to a central streamline (similar to rifling). In-plane autorotation is likely to be the predominant mode for particles with a multiplicity of symmetrically-aligned arms or surfaces radiating about a central axis. Such particles are likely to closely follow streamlines of flow unless they are influenced by secondary flows and turbulence of significant magnitude or are hindered by physical obstructions. This motion is likely to persist until the flow velocity reduces to levels equivalent to or below the counteracting drag forces, as is the case with sedimentation under zero flow conditions. In vivo, such particles may have a higher probability of exhibiting a deep lung deposition pattern because of their ability to follow streamlines.

Spiral autorotation is likely for particles with multiple radial surfaces that are asymmetrically aligned about a central axis, thereby creating an off-axis center of gravity (“CG”). The radius of the spiral is proportional to the magnitude of the eccentricity of the CG from the central axis. However, spiraling particles are likely to be loosely bound to the flow streamlines (because of increased centrifugal force) and are more susceptible to changes in flow velocity, secondary flows and turbulence. While this motion is likely to persist until zero flow conditions, there is a higher probability that with respect to pulmonary deposition, such particles will impact on airway walls of smaller diameters than that of their defining spiral.

Asymmetrical particles without radiating arms are likely to exhibit “tumbling” as their primary mode of flight. Particles in which the CG is offset from the longitudinal axis representing the flow streamline (usually the z-axis) but are balanced in the plane perpendicular to flow are likely to predominantly tumble about one of the remaining two axes. However, truly asymmetrical particles, in which the CG is offset from all three axes, are likely to exhibit a complex tumbling mechanism. Pulmonary deposition patterns for these particles are relatively unpredictable as they are not likely to follow streamlines very closely. As a result, these are likely to impact easily in large airways, at bifurcations and around obstructions in the respiratory tract, i.e., predominantly in the upper lungs. Deposition patterns for such particles can be predicted by sophisticated computation fluid dynamics (CFD) modeling software and verified by high-resolution pulmonary imaging using MRI, CT and other radio-labeled imaging modalities.

Lift-generating designs may incorporate aerofoil surfaces, streamlined edges or other features that may increase the particles' ability to remain afloat longer in low Reynold's number (Re) laminar flow regimes. They may also be designed to induce leading edge vortices, which further stabilize the particles and generate additional lift.²² Such particles are likely to exhibit stable and streamlined flight patterns and increased settling times in zero flow sedimentation conditions. These particles are again more likely to deposit in small airways and terminal bronchioles than spherical particles of an equivalent aerodynamic diameter.

In one embodiment, the particles exhibit autorotation, tumbling, and/or generation of lift, a combination of two or more of these characteristics, or all three. However, it is possible to envision more sophisticated designs by the use of surfaces (e.g., aerofoils, fins, stabilizers, etc.), fenestrations, surface modifications (e.g., grooves, ridges, stealthing agents. Etc.), compositions and/or external control mechanisms (e.g., magnetic fields for instance) that provide additional lift, stabilization, streamlining or flight control. These may be used independently or in combination in order to predispose particles to a single mode of flight, flow regime or in the case of therapeutic applications, a targeted anatomical location.

With regard to particular embodiments of the present invention, the ellipsoid and Lorenz-shaped particles may be configured to promote both autorotation and off-axis tumbling. In one embodiment, the Lorenz particles are configured to promote tumbling about a single axis (see e.g., FIG. 3C), while the introduction of a fenestration into the Lorenz particular facilitates tumbling about two axes (see e.g., FIG. 3D). The ellipsoid-shaped particles may be configured for similar flight altering characteristics (i.e., affecting both autorotation and tumbling), wherein some ellipsoid particles promote tumbling about one axis (see e.g., FIG. 3G) or two axes (see e.g., FIG. 3G). Moreover, a symmetrically-shaped particle (e.g., a radially aligned particle such as shown in FIG. 3F) may promote autorotation about a central axis. In addition, the ball-and-stick configurations may promote tumbling but not autorotation (see e.g., FIGS. 3J, 3M and 3N).

Thus, each particle may be configured to promote one or more flight characteristic regardless of the shape chosen, as well as increase the settling time of the particle in comparison to spherical and other standard-shaped particles (see Example 6 below). The particle can be symmetric and have an on-axis center of mass or be asymmetric and have an off-axis center of mass. The particles may have a specific asymmetrical shape or include additional features to enhance its asymmetrical properties. For example, the particle can be fenestrated to create unbalanced CGs and thereby induce autorotation, tumbling, and/or lift. Mass could also be added to the particle for enhancing asymmetry (see e.g., FIG. 3J where the rounded portion 14 is employed to add additional mass). Moreover, the particles may promote modulation of matrix anisotropy by redistributing density in specific directions. Furthermore, the leading or trailing edge of a particle may include different cross sections (e.g., rectangular) and can be modified to facilitate lift, such as by using a particle having an airfoil cross section or lift-generating surface. As such, any number of factors may be customized for a particular particle in order to modify the normal flight characteristics. Such modification of the normal flight characteristics can increase the settling time, thereby altering the delivery of the particles in the airstream. By increasing time of flight, the probability that these particles deposit deeper in the lung also increases. Additionally, the particles may be influenced by secondary flows at airway bifurcations (in normal lungs) and obstructed airways (in case of COPD and asthma, for instance). It is expected that this would lead to anatomically differential deposition and to target specific airway generations and regions of the respiratory tract.

2) Evaluation of the Aerosolization Characteristics of the Engineered Aerosols.

The aerosol particles will be analyzed by a number of techniques including particle scattering techniques, 8-stage Anderson Cascade Impactor analyses²⁰, stereo digital particle image velocimetry (DPIV)¹⁹ to measure the 3D velocity field and computational fluid dynamics. The Anderson Cascade Impactor allows the aerosolization analysis of dry powders. The technique involves the aerosolization of particles into a series of baffles where various particles can be collected at each of the stages. The mass collected at each stage depends upon the orifice velocity of the specific stage, the distance between orifices, the collection surface and the collection characteristics of the preceding stage. The combination of constant flow rate and successively smaller orifices in each of the stages increases the velocity of the sample air as it flows through the impactor resulting in impaction of progressively smaller particles in subsequent stages. The aerodynamic size distribution can then be determined by quantifying the mass fraction of particles found at the various stages. We will also measure the three-dimensional flow around dynamically scaled models of the new particle designs using DPIV.¹⁹

3) Engineered Aerosol Particles for the Delivery of Therapeutics to the Lung and to the CNS.

Pulmonary drug delivery routes have many advantages over other methods for both local and systemic delivery. For example, inhalation of therapeutics allows targeted delivery of high concentrations of drug for treatment of respiratory diseases while limiting systemic toxicity. Alternatively, the large surface area and high solute permeability of the lung can provide a non-invasive route for systemic absorption of therapeutics and biologics (for example, peptides and proteins) that cannot be delivered orally or have poor therapeutic efficacy when delivered systemically. However, current methods for inhaled drug delivery are compromised by inefficient delivery systems and limitations imposed by the individual physiochemical properties of each drug. There are challenges in “particulating” many of the existing and emerging new drugs including many insoluble small molecules and biologicals (siRNA, antibodies). Monoclonal antibodies are delivered today only via injections or intravenous infusions. There is significant interest in alternative delivery routes for biological therapeutics. Nasal delivery is attractive because of its convenience and the large surface area for absorption generated by the nasal microvilli. Direct routes of administration of biological therapeutics to the brain that are non-invasive via transnasal routes²¹ are also highly desirable, especially therapeutics for pain management, cystic fibrosis and the management of diabetes.

It is envisioned that the autorotation of engineered particles may dramatically change the flight characteristics of particles in the pulmonary system opening up opportunities to access the deep lung and direct access to the CNS via intranasal routes. Initial screening of particle deposition and clearance will be studied in the nasal passages of a rat model using planar gamma scintigraphy of Tc99m labeled particles. After identifying particle shapes and sizes that are deemed best at effecting the desire location of deposition, subsequent studies may be designed to assess clearance from the lower respiratory tract utilizing and comparing gamma (planar and SPECT), positron emission (PET), and magnetic resonance (MRI) imaging. Gadolinium is highly paramagnetic and has a profound effect on the longitudinal relaxation of water protons leading to a hyperintense signal in magnetic resonance imaging. Gd³⁺ ion will be attached to our particles via a multidentate ligand such as DOTA (1,4,7,10-tetraazacyclododecane-N,N′,N″,N′″-tetraacetic acid) or DTPA (diethylenetriamine pentaacetic acid). Technetium is a short-lived gamma-emitting nuclear isomer 99mTc and is used in nuclear medicine for a wide variety of diagnostic tests including SPECT imaging. ⁶⁴Cu is a long-lived positron emitter useful for micro PET/CT imaging. Similar to gadolinium, ⁶⁴Cu and technetium can be complexed with the mulitdentate ligands.

In some embodiments of the invention, the engineered particles of the invention carry one or more therapeutic agents as the cargo packaged therein or attached thereto. Where the engineered particle includes at least one therapeutic agent as the cargo, it is recognized that a single agent or a combination of agents may be contained within the same engineered particle. Thus, in some instances, the engineered particles of the invention are a homogeneous mix of particles; that is, a mixture of particles containing the same cargo or agent(s). Alternatively, a composition of engineered particles of the invention may comprise a heterogeneous mixture of particles. That is engineered particles containing different cargo or agents may be mixed and administered to a subject in need thereof.

Depending upon their intended therapeutic use, the engineered particles of the invention can comprise one or more therapeutic agents of interest. Such agents include but are not limited to small molecule pharmaceuticals, therapeutic and diagnostic proteins, antibodies, DNA and RNA sequences, imaging agents, and other active pharmaceutical ingredients. Active agents include the active agent proteins listed above. Active agents also include, without limitation, analgesics, anti-inflammatory agents (including NSAIDs), anticancer agents, antimetabolites, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, therapeutic proteins, enzymes, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, and antiviral agents.

Anticancer agents include, without limitation, alkylating agents, antimetabolites, natural products, hormones, topoisomerase I inhibitors, topoisomerase II inhibitors, RNA/DNA antimetabolites, DNA antimetabolites, antimitotic agents and antagonists, and miscellaneous agents, such as radiosensitizers. Examples of alkylating agents include, without limitation, alkylating agents having the bis-(2-chloroethyl)-amine group such as chlormethine, chlorambucile, melphalan, uramustine, mannomustine, extramustinephoshate, mechlore-thaminoxide, cyclophosphamide, ifosfamide, and trifosfamide; alkylating agents having a substituted aziridine group such as tretamine, thiotepa, triaziquone, and mitomycine; alkylating agents of the alkyl sulfonate type, such as busulfan, piposulfan, and piposulfam; alkylating N-alkyl-N-nitrosourea derivatives, such as carnustine, lomustine, semustine, or streptozotocine; and alkylating agents of the mitobronitole, dacarbazine, and procarbazine type. See, for example U.S. Pat. No. 5,399,363. Antimitotic agents include allocolchicine, halichondrin B, colchicine, dolastatin, maytansine, rhizoxin, taxol and taxol derivatices, paclitaxel, vinblastine sulfate, vincristine sulfate, and the like. Topoisomerase I inhibitors include camptothecin, aminocamptothecin, camptothecin derivatives, morpholinodoxorubicin, and the like. Topoisomerase II inhibitors include doxorubicin, amonafide, m-AMSA, anthrapyrazole, pyrazoloacridine, daunorubicin, deoxydoxorubicin, mitoxantrone, menogaril, N,N-dibenzyl daunomycin, oxanthrazole, rubidazone, and the like. Other anticancer agents can include immunosuppressive drugs, such as cyclosporine, azathioprine, sulfasalazine, methoxsalen, and thalidomide.

Antimetabolites include, without limitation, folic acid analogs, such as methotrexate; pyrimidine analogs such as fluorouracil, floxuridine, tegafur, cytarabine, idoxuridine, and flucytosine; and purine derivatives such as mercaptopurine, thioguanine, azathioprine, tiamiprine, vidarabine, pentostatin, and puromycine. Antibiotics also include gentamicin, kanamycin, neomycin, netilmicin, streptomycin, tobramycin, paromomycin, geldanamycin, herbimycin, loracarbef, ertapenem, doripenem, imipenem, cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefdinir, cefepime, teicoplanin, vancomycin, azithromycin, clarithromycin, cirithromycin, erythromycin, roxithromycin, troleandomycin, telithromycin, spectinomycin, aztreonam, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, bacitracin, colistin, polymyxin B, ciprofloxacin, enoxacin, gatifloxacin, levofloxacin, lomeflxacin, moxifloxacin, norfloxacin, ofloxacin, trovafloxacin, mafenide, prontosil, sulfacetamide, sulfamethizole, sulfanilamide, sulfasalazine, sulfisoxazole, trimethoprim, trimethoprim-sulfamethoxazole, demeclocycline, doxycycline, minocycline, oxytetracycline, tetracycline, arsphenamine, chloramphenicol, clindamycin, lincomycin, ethambutol, fusfomycin, fusidic acid, furazolidone, isoniazid, linezoilid, metronidazole, mupirocin, nitrofurantoin, platensimycin, pyrazinamide, quinupristin, dalfopristin, rifampin, rifampicin, tinidazole, etc.

Therapeutic proteins include enzymes, blood factors, blood clotting factors, insulin, erythropoietin, interferons, including interferon-α, interferon-β, protein C, hirudin, granulocyte-macrophage colony-stimulating factor, somatropin, epidermal growth factor, albumin, hemoglobin, lactoferrin, angiotensin-converting enzyme, glucocerebrosidase, human growth hormone, VEGF, and the like. Proteins also include antigenic proteins or peptides. Proteins of interest also include, without limitation, enzymes, growth factors, monoclonal antibody, antibody fragments, single-chain antibody, immunoglobulins, clotting factors, amylase, lipase, protease, cellulose, urokinase, galactosidase, staphylokinase, hyaluronidase, tissue plasminogen activator, and the like. Therapeutic proteins can include monoclonal antibodies, for example abciximab, adalimumab, alemtuzumab, basiliximab, bevacizumab, cetuximab, daclizumab, eculizumab, efalizumab, herceptin, britumomab tiuxetan, infliximab, muromonab-CD3, natalizumab, omalizumab, palivizumab, panitumumab, ranibizumab, rituximab, traztuzumab, etc.

Examples of natural products include vinca alkaloids, such as vinblastine and vincristine; epipodophylotoxins, such as etoposide and teniposide; antibiotics, such as adriamycine, daunomycine, doctinomycin, daunorubicin, doxorubicin, mithramycin, bleomycin, and mitomycin; enzymes, such as L-asparaginase; biological response modifiers, such as alpha-interferon; camptothecin; taxol; and retinoids, such as retinoic acid.

Other agents include, without limitation, MR imaging agents, contrast agents, gadolinium chelates, gadolinium-based contrast agents, radiosensitizers, such as, for example, 1,2,4-benzotriazin-3-amine 1,4-dioxide (SR 4889) and 1,2,4-benzotriazine-7-amine 1,4-dioxide (WIN 59075); platinum coordination complexes such as cisplatin and carboplatin; anthracenediones, such as mitoxantrone; substituted ureas, such as hydroxyurea; and adrenocortical suppressants, such as mitotane and aminoglutethimide.

In some embodiments, the engineered particles of the invention comprise one or more therapeutic agents that are to be administered to a subject via the lungs or to the central nervous system.

In this manner, following their generation within a patterned template or mold via PRINT®, the engineered particles of the invention having the desired particle size and shape that provide auto-rotation, tumbling, and/or lift when entrained in an airstream, and comprising one or more therapeutic agents of interest, can be released from the patterned template and used to deliver therapeutic agents to the lung via pulmonary inhalation and to the central nervous system via intranasal administration. In some embodiments, the releasing of the one or more particles is performed by one of: (a) applying the patterned template to a substrate, wherein the substrate has an affinity for the one or more particles; (b) deforming the patterned template such that the one or more particles is released from the patterned template; (c) swelling the patterned template with a first solvent to extrude the one or more particles; (d) washing the patterned template with a second solvent, wherein the second solvent has an affinity for the one or more particles; and (e) applying a mechanical force to the one or more particles. In some embodiments, the mechanical force is applied by contacting one of a Doctor blade and a brush with the one or more particles. In some embodiments, the mechanical force is applied by ultrasonics, megasonics, electrostatics, or magnetic means. In some embodiments, the method comprises harvesting or collecting the nanoparticles. In some embodiments, the harvesting or collecting of the particles comprises a process selected from the group consisting of scraping with a doctor blade, a brushing process, a dissolution process, an ultrasound process, a megasonics process, an electrostatic process, and a magnetic process.

The preferred size for the engineered particles of the invention when they are to be delivered via pulmonary inhalation is less than about 10.0 μm mean diameter, less than about 7.0 μm, or less than about 6.0 μm mean diameter. In other embodiments, the particle sizes are in the range of 0.1 to 5.0 μm, or in the range of about 1.0 to about 5.0 μm mean diameter.

In this manner, the engineered particles of the invention carrying one or more therapeutic agents of interest packaged therein or attached thereto are formulated as compositions for pulmonary inhalation or intranasal administration. By “pulmonary inhalation” is intended the composition comprising the engineered particles are directly administered to the lung by delivering the particles in an aerosol or other suitable preparation from a delivery device into the oral cavity of the subject as the subject inhales through the oral cavity. By “aerosol” is intended a suspension of solid or liquid particles in flowing air or other physiologically acceptable gas stream. Other suitable preparations include, but are not limited to, mist, vapor, or spray preparations. Pulmonary inhalation could also be accomplished by other suitable methods known to those skilled in the art. These may include liquid instillation using a suitable device or other such methods. Pulmonary inhalation results in deposition of the inhaled engineered particles deep into the lungs or alveolar region of the subject's lungs. Depending upon the size and shape, and material from which they are formed, the engineered particles can be designed to ensure deposition deep within the lung for treatment of local respiratory infection or disease while limiting systemic delivery. Alternatively, the engineered particles can be designed such their size, shape, and/or material from which they are formed provides for absorption, passively or actively, across the alveoli epithelium and capillary epithelium layers into the bloodstream for subsequent systemic distribution of the cargo, i.e., the one or more therapeutic agents packaged therein or attached thereto.

Pulmonary administration of the engineered particles of the invention requires dispensing of the engineered particles from a delivery device into the oral cavity of a subject during inhalation. For purposes of the present invention, compositions comprising the engineered particles of the invention are administered via inhalation of an aerosol or other suitable preparation that is obtained from an aqueous or nonaqueous solution or suspension form, or a solid or dry powder form of the composition, depending upon the delivery device used. Such delivery devices are well known in the art and include, but are not limited to, nebulizers, metered-dose inhalers, and dry powder inhalers, or any other appropriate delivery mechanisms that allow for dispensing of a composition as an aqueous or nonaqueous solution or suspension or as a solid or dry powder form. By “aqueous” is intended a composition prepared with, containing, or dissolved in water, including mixtures wherein water is the predominating substance in the mixture. A predominating substance is present in a greater quantity than another component of the mixture. By “nonaqueous” is intended a composition prepared with, containing, or dissolved in a substance other than water or mixtures wherein water is not the predominating substance in the mixture. By “solution” is intended a homogeneous preparation of two or more substances, which may be solids, liquids, gases, or intercombinations thereof. By “suspension” is intended a mixture of substances such that one or more insoluble substances are homogeneously dispersed in another predominating substance.

For purposes of the present invention, the terms “solid” and “dry powder” are used interchangeably. By “solid” or “dry powder” form of a composition is intended the composition has been dried to a finely divided powder having a moisture content below about 10% by weight, usually below about 5% by weight, and preferably below about 3% by weight. This dry powder form of the composition consists of engineered particles of the invention, which comprise one or more therapeutic agents of interest as cargo. In some embodiments, the particle sizes are less than about 10.0 μm mean diameter, less than about 7.0 μm, or less than about 6.0 μm mean diameter. In other embodiments, the particle sizes are in the range of 0.1 to 5.0 μm, or in the range of about 1.0 to about 5.0 μm mean diameter.

Thus, the harvested engineered particles of the invention intended for use in the pulmonary delivery methods of the present invention may either be formulated as a liquid solution or suspension in the delivery device, for example, a nebulizer, or first be processed into a dry powder form using a lyophilization technique well known in the art. Alternatively, the harvested engineered particles of the invention comprising one or more therapeutic agents can be formulated as a liquid solution or suspension and then processed into a dry powder form using, for example, lyophilization. As yet another alternative, the engineered particles of the invention can be prepared as a thin film that can then be placed within a delivery device that allows for pulsed release of the engineered particles, for example, by vibration of the film surface, into the airways of the lungs.

Where a liquid solution or suspension is used in the delivery device, a nebulizer, a metered dose inhaler, or other suitable delivery device delivers, in a single or multiple fractional dose, by pulmonary inhalation a therapeutically effective amount of the engineered particles to the subject's lungs as droplets, preferably having the same particle size range noted above for the dry powder form. By “therapeutically effective amount” is intended an amount of the engineered particles that provides for the release of the one or more therapeutic agents in an amount that is useful in the treatment, prevention, or diagnosis of a disease or condition. The liquid solution or suspension of the composition may be used with physiologically appropriate stabilizing agents, excipients, bulking agents, surfactants, or combinations thereof. Examples of suitable excipients include, but are not limited to, buffers, viscosity modifiers, or other therapeutically inactive but functional additives.

Where the engineered particles comprising one or more therapeutic agents of interest are prepared in lyophilized form prior to use in the pulmonary delivery methods of the invention, the lyophilized composition is processed to obtain a finely divided dry powder comprising the engineered particles having the desirable sizes and shapes to provide at least one of auto-rotation and lift through creation of a leading edge vortex when entrained in an airstream.

The resulting dry powder form of the particle-containing composition is then placed within an appropriate delivery device for subsequent preparation as an aerosol or other suitable preparation that is delivered to the subject via pulmonary inhalation. Where the dry powder form of the particle-containing composition is to be prepared and dispensed as an aqueous or nonaqueous solution or suspension, a metered-dose inhaler, or other appropriate delivery device is used. A therapeutically effective amount of the dry powder form of the particle-containing composition is administered in an aerosol or other preparation suitable for pulmonary inhalation. The amount of dry powder form of the particle-containing composition placed within the delivery device is sufficient to allow for delivery of a therapeutically effective amount of the engineered particles to the subject by inhalation. Thus, the amount of dry powder form to be placed in the delivery device will compensate for possible losses to the device during storage and delivery of the dry powder form of the composition. Following placement of the dry powder form within a delivery device, the engineered particles are suspended in an aerosol propellant. The pressurized nonaqueous suspension is then released from the delivery device into the air passage of the subject while inhaling. The delivery device delivers, in a single or multiple fractional dose, by pulmonary inhalation a therapeutically effective amount of the engineered particles to the subject's lungs. The aerosol propellant may be any conventional material employed for this purpose, such as a chlorofluorocarbon, a hydrochloro-fluorocarbon, a hydrofluorocarbon, or a hydrocarbon, including trichlorofluoromethane, dichlorodifluro-methane, dichlorotetrafluoromethane, dichlorodifluoro-methane, dichlorotetrafluoroethanol, and 1,1,1,2-tetra-fluoroethane, or combinations thereof. A surfactant may be added to the composition to reduce adhesion of the particle-containing dry powder to the walls of the delivery device from which the aerosol is dispensed. Suitable surfactants for this intended use include, but are not limited to, sorbitan trioleate, soya lecithin, and oleic acid. Devices suitable for pulmonary delivery of a dry powder form of a composition as a nonaqueous suspension are commercially available. Examples of such devices include the Ventolin metered-dose inhaler (Glaxo Inc., Research Triangle Park, N.C.) and the Intal Inhaler (Fisons, Corp., Bedford, Mass.). See also the aerosol delivery devices described in U.S. Pat. Nos. 5,522,378, 5,775,320, 5,934,272 and 5,960,792, herein incorporated by reference.

Where the solid or dry powder form of the particle-containing composition is to be delivered as a dry powder form, a dry powder inhaler or other appropriate delivery device may be used. The dry powder form of the particle-containing composition is preferably prepared as a dry powder aerosol by dispersion in a flowing air or other physiologically acceptable gas stream in a conventional manner. Examples of commercially available dry powder inhalers suitable for use in accordance with the methods herein include the Spinhaler powder inhaler (Fisons Corp., Bedford, Mass.) and the Ventolin Rotahaler (Glaxo, Inc., Research Triangle Park, N.C.). See also the dry powder delivery devices described in WO 93/00951, WO 96/09085, WO 96/32152, and U.S. Pat. Nos. 5,458,135, 5,785,049, and 5,993,783, herein incorporated by reference.

The dry powder form of the particle-containing composition can be reconstituted to an aqueous solution for subsequent delivery as an aqueous solution aerosol using a nebulizer, a metered dose inhaler, or other suitable delivery device. In the case of a nebulizer, the aqueous solution held within a fluid reservoir is converted into an aqueous spray, only a small portion of which leaves the nebulizer for delivery to the subject at any given time. The remaining spray drains back into a fluid reservoir within the nebulizer, where it is aerosolized again into an aqueous spray. This process is repeated until the fluid reservoir is completely dispensed or until administration of the aerosolized spray is terminated. Such nebulizers are commercially available and include, for example, the Ultravent nebulizer (Mallinckrodt Inc., St. Louis, Mo.) and the Acorn II nebulizer (Marquest Medical Products, Englewood, Colo.). See also the nebulizer described in WO 93/00951, and the device for delivering aerosolized aqueous formulations described in U.S. Pat. No. 5,544,646; herein incorporated by reference.

In accordance with the method of the present invention, the aqueous or nonaqueous solution or suspension or solid or dry powder form of the composition comprising the engineered particles having one or more therapeutic agents as cargo is administered to a subject in the form of an aerosol or other preparation suitable for pulmonary inhalation. By “subject” is intended any animal. Preferably the subject is mammalian, most preferably the subject is human. Mammals of particular importance other than human include, but are not limited to, dogs, cats, cows, horses, sheep, and pigs.

The engineered particles of the invention, when formulated for pulmonary delivery, find use in the treatment of a variety of conditions. As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, stabilized (i.e., not worsening) state of disease, preventing or delaying spread (e.g., metastasis) of disease, preventing or delaying occurrence or recurrence of disease, delay or slowing of disease progression, amelioration of the disease state, and remission (whether partial or total). Also encompassed by “treatment” is a reduction of pathological consequence of a disease. The methods of the invention contemplate any one or more of these aspects of treatment. In this manner, the engineered particles of the invention can be designed to comprise one or more therapeutic agents useful for pulmonary delivery of vaccines and treatments for bacterial infections, cystic fibrosis, emphysema, and lung cancer, for example. Alternatively, the engineered particles of the invention may comprise one or more therapeutic agents for systemic delivery via pulmonary inhalation, for example, any of the therapeutic agents described elsewhere herein.

In some embodiments, the therapeutic agents to be delivered via pulmonary inhalation include therapeutic, prophylactic, and/or diagnostic agents for treatment of respiratory infectious diseases such as TB, severe acute respiratory syndrome (SARS), influenza, and small pox. Suitable therapeutic agents include agents that can act locally, systemically or a combination thereof. Examples of therapeutic agents include, but are not limited to, synthetic inorganic and organic compounds, proteins, peptides, polypeptides, DNA and RNA nucleic acid sequences, or any combination or mimic thereof, having therapeutic, prophylactic, or diagnostic activities.

In some of these embodiments, the engineered particles of the invention provide for pulmonary delivery of one or more therapeutic agents selected from the group consisting of an antibiotic for treatment of a respiratory infection such as tuberculosis, such as capreomycin, PA-824, rifapicin, rifapentine, and quinolones (e.g. Moxifloxacin (BAY 12-8039), aparfloxacin, gatifloxacin, CS-940, Du-6859a, sitafloxacin, HSR-903, levofloxacin, WQ-3034), ciprofloxacin, and levofloxacin. Capreomycin is a relatively hydrophilic antibiotic molecule. It is currently used as a second-line defense molecule, in the prevention of TB. Capreomycin shows a one to two log decrease in colony forming units (“CFU”) after one month against non-replicating TB in vitro, so there is potential for latent TB treatment, as reported by Heifets, et al. Ann. Clin. Microbiol. Antimicrobiol. 4(6) (2005). PA-824 is a bactericidal antibiotic which targets a flavenoid F420 and also prevents mycolic acid synthesis and lipid biosynthesis. Rifapentine inhibits RNA polymerase by binding to the beta-subunit of the protein and acts as a bactericidal antibiotic. In yet other embodiments, the therapeutic agent is a vaccine, such as a BCG vaccine, which is effective against TB, or flu antigens.

For treatment of viral respiratory infections, the therapeutic agent(s) packaged within or attached to the engineered particles of the invention is preferably an antiviral alone or in combination with a vaccine. Four antiviral medications are commonly prescribed for the A category of influenza viruses, amantadin, rimantadine, zanamavir and the widely-stockpiled oseltamivir. These are neuraminidase inhibitors, which block the virus from replicating. If taken within a couple of days of the onset of illness, they can ease the severity of some symptoms and reduce the duration of sickness.

Multi-drug resistant tuberculosis (MDR-TB) is emerging as a significant public health threat, creating an unmet medical need that requires the development of new treatment approaches. In a preferred embodiment very high drug doses are delivered to the site of primary infection for rapid sterilization of the lung mucosa and reduction in the duration of MDR-TB therapy. The formulation for treatment of drug resistant forms of infection may include very high loading of one or more antibiotics or a combination of antibiotic and vaccine.

The engineered particle composition can be administered by pulmonary inhalation to treat other conditions of the respiratory tract, including, but not limited to, pulmonary fibrosis, bronchiolitis obliterans, lung cancer (for example, non-small cell lung cancer of the squamous cell carcinoma, adenocarcinoma, and large cell carcinoma types, and small cell lung cancer), bronchioalveolar carcinoma, and the like.

In other embodiments of the invention, the engineered particles of the invention comprise one or more agents for administration to the central nervous system via intranasal delivery. Thus, the engineered particles of the invention can comprise one or more therapeutic agents for administration into the nasal cavity, preferably deep within the nasal cavity, to allow for entry into the central nervous system along olfactory sensory neurons to yield significant concentrations in the cerebral spinal fluid and olfactory bulb. Such therapeutics include, for example, those suitable for pain management, and treatment of neurodegenerative disorders. The engineered particles of the invention can be administered intranasally to deliver agents to the brain for diagnosis, treatment or prevention of disorders or diseases of the CNS, brain, and/or spinal cord. These disorders can be neurologic or psychiatric disorders. These disorders or diseases include brain diseases such as Alzheimer's disease, Parkinson's disease, Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia. The engineered particles of the invention can be delivered intranasally to subjects suffering from or at risk for nerve damage from cerebrovascular disorders such as stroke in the brain or spinal cord, from CNS infections including meningitis and HIV, from tumors of the brain and spinal cord, or from a prion disease. The engineered particles of the invention can be administered intranasally to deliver agents to counter CNS disorders resulting from ordinary aging (e.g., anosmia or loss of the general chemical sense), brain injury, or spinal cord injury.

Thus, in some embodiments, the engineered particles of the invention can comprise GM-1 ganglioside, fibroblast growth factor, particularly basic fibroblast growth factor (bFGF), insulin-like growth factor, particularly insulin-like growth factor-I (IGF-I), nerve growth factor (NGF), phosphatidylserine, a cytokine, such as an interferon, an interleukin, or a tumor necrosis factor, plasmid or vector, or a polynucleotide, and the like. The polynucleotide may be provided as an antisense agent or interfering RNA molecule such as an RNAi or siRNA molecule to disrupt or inhibit expression of an encoded protein. siRNA includes small pieces of double-stranded RNA molecules that bind to and neutralize specific messenger RNA (mRNA) and prevent the cell from translating that particular message into a protein. Alternatively, the polynucleotide may comprise a sequence encoding a peptide or protein of interest such as a therapeutic protein or antigenic protein or peptide. Accordingly, the polynucleotide may be any nucleic acid including but not limited to RNA and DNA. The polynucleotides may be of any size or sequence and may be single- or double-stranded. Methods for synthesis of RNA or DNA sequences are known in the art. See, for example, Ausubel et al. (1999) Current Protocols in Molecular Biology (John Wiley & Sons, Inc., NY); Sambrook et al. (1989) Molecular Cloning: A Laboratory Manual (2nd ed.) (Cold Spring Harbor Laboratory Press, Plainview, N.Y.); herein incorporated by reference.

The engineered particles comprising one or more therapeutic agents of interest can be suspended in a biocompatible medium to form a pharmaceutical composition for intranasal administration. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The pharmaceutical composition of the invention can include other agents, excipients, or stabilizers. For example, to increase stability by increasing the negative zeta potential of the engineered particles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-.alpha.-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

4) Incorporation of Sensing, Signaling, and Taggant Capabilities onto Engineered Aerosol Particles.

According to some embodiments, engineered aerosol particles may be modified with chemical and biological recognition agents and develop. Further, high sensitivity strategies for readout may be developed. In particular, libraries of particles with ideal aerosolization characteristics may be generated in an effort to diagnose the nature and threat level of chemical/biological plumes at a distance. For example, PRINT® particles may be “structured” with various components in various regions that can be used in a multi-plexed manner for signal detection. In addition, these structures can be used as nanoscopic labels to covertly track the movement of personnel and materials. According to some aspects, the auto-rotating aerosol particles may be loaded with RFIDs.

In some embodiments, the engineered particles may further comprise one or more cargos. Cargo may include various substances, materials, or other objects of interest. In some instances, the term cargo refers to a therapeutic. A therapeutic can include a small molecule, biologic, or other substance utilized for the treatment or detection of disease. Therapeutic cargos may include but are not limited to small molecule pharmaceuticals, therapeutic and diagnostic proteins, antibodies, DNA and RNA sequences, imaging agents, and other active pharmaceutical ingredients. Further, such cargo may include active agents which may include, without limitation, analgesics, anti-inflammatory agents (including NSAIDs), anticancer agents, antimetabolites, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, therapeutic proteins, enzymes, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, and antiviral agents. The cargo may include a polynucleotide. The polynucleotide may be provided as an antisense agent or interfering RNA molecule such as an RNAi or siRNA molecule to disrupt or inhibit expression of an encoded protein. In some embodiments, the cargo may comprise additional components, including drugs, such as anticancer agents, e.g., nitrogen mustard, cisplatin, and doxorubicin; targeting ligands, such as cell-targeting peptides, cell-penetrating peptides, integrin receptor peptide (GRGDSP), melanocyte stimulating hormone, vasoactive intestional peptide, anti-Her2 mouse antibodies, and a variety of vitamins; viruses, polysaccharides, cyclodextrins, proteins, liposomes, anthracenediones, such as mitoxantrone; substituted ureas, such as hydroxyurea; and adrenocortical suppressants, such as mitotane and aminoglutethimide and borate nanoparticles to aid in boron neutron capture therapy (BNCT) targets.

In some embodiments, the term cargo may refer to a component that can incorporate sensing, signaling, or taggant capabilities onto the engineered nanoparticles. Cargo may include, without limitation, MR imaging agents, contrast agents, gadolinium chelates, gadolinium-based contrast agents, radiosensitizers, such as, for example, 1,2,4-benzotriazin-3-amine 1,4-dioxide (SR 4889) and 1,2,4-benzotriazine-7-amine 1,4-dioxide (WIN 59075); and optical nanoparticles, such as CdSe for optical applications.

According to other embodiments, the engineered aerosol particles may be capable of carrying other particles, which, in some instances, may be smaller, therewith. For example, a microparticle may carry one or more nanoparticles therein to the delivery site, wherein the nanoparticles may permeate or otherwise diffuse through the microparticle (e.g., through a membrane).

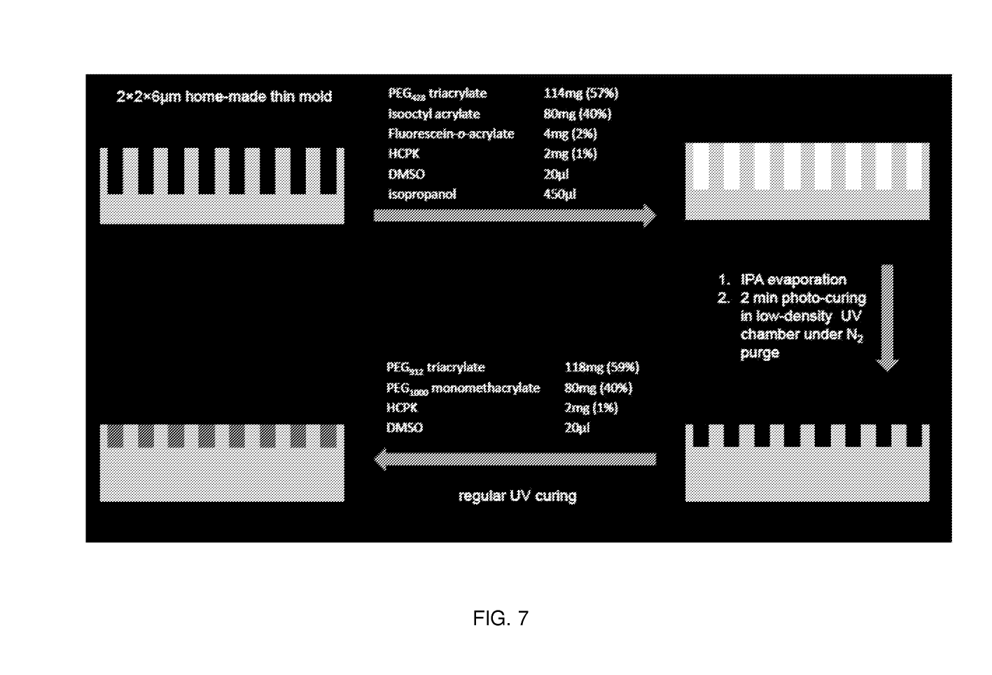

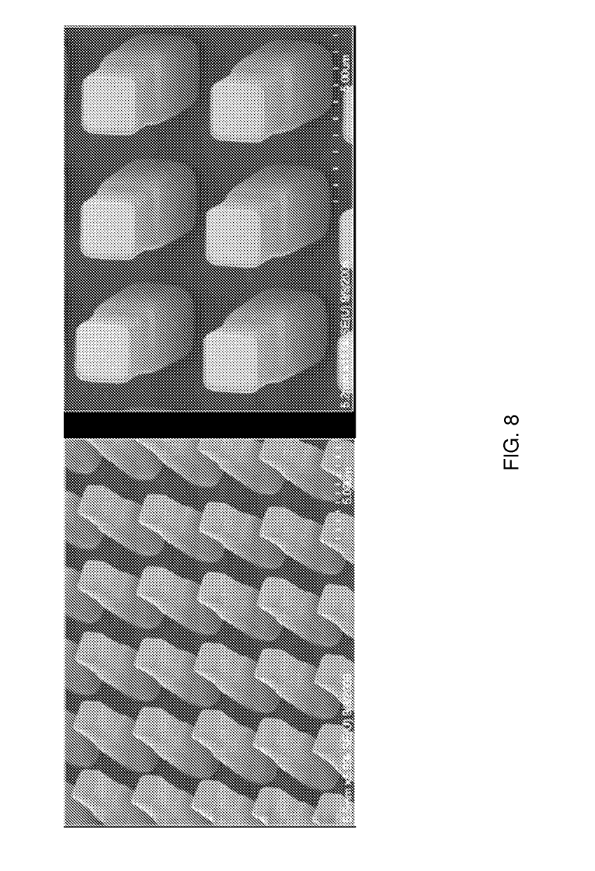

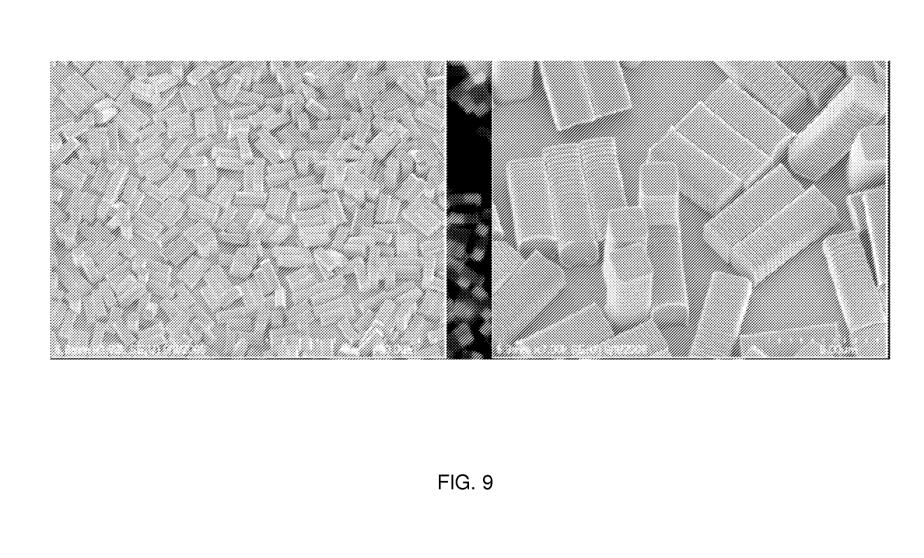

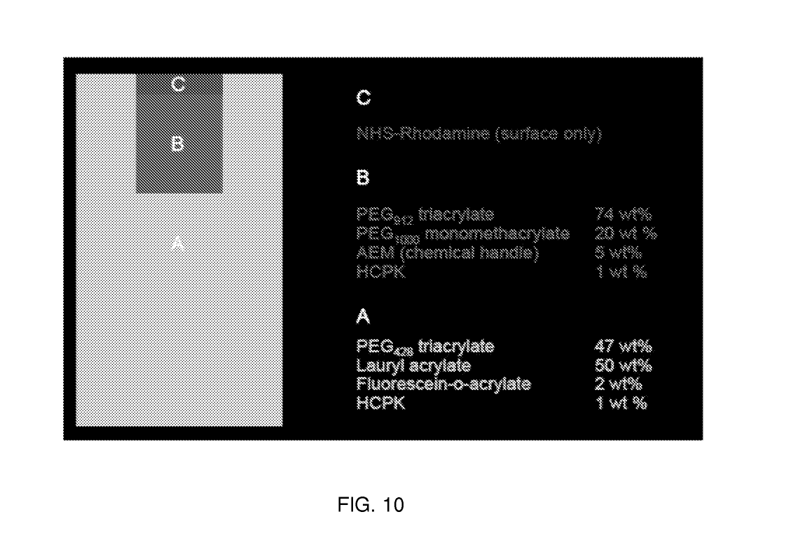

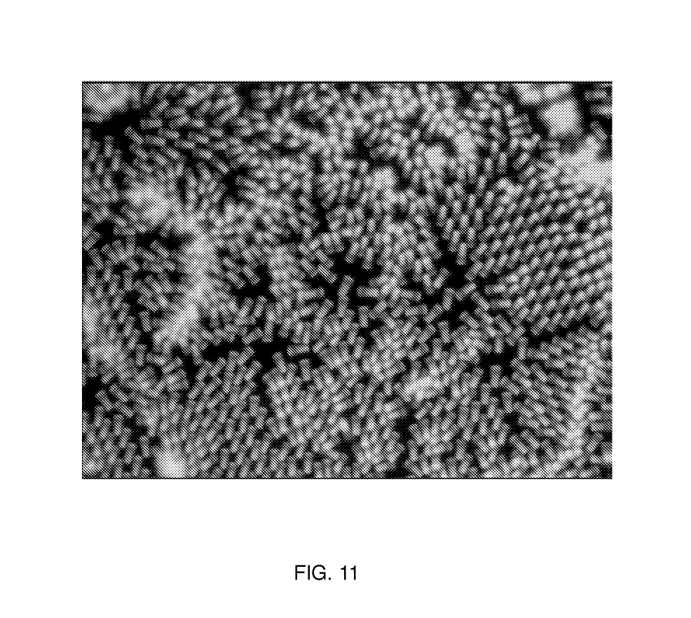

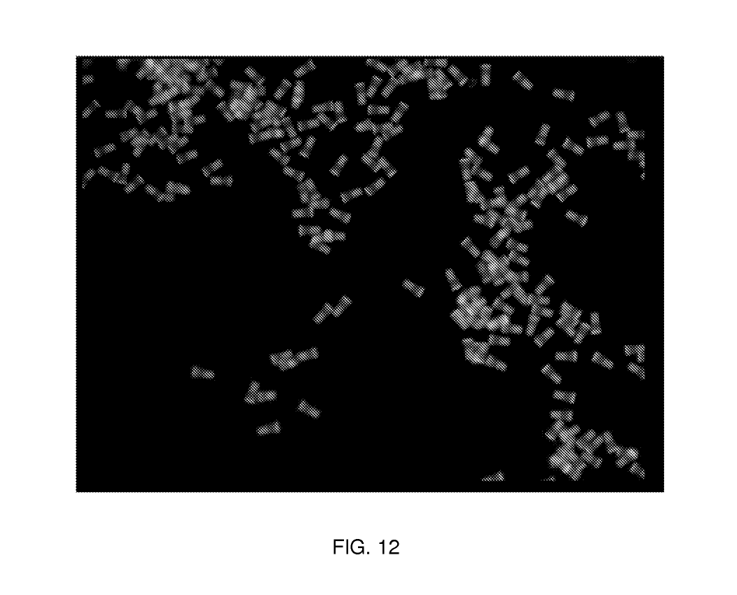

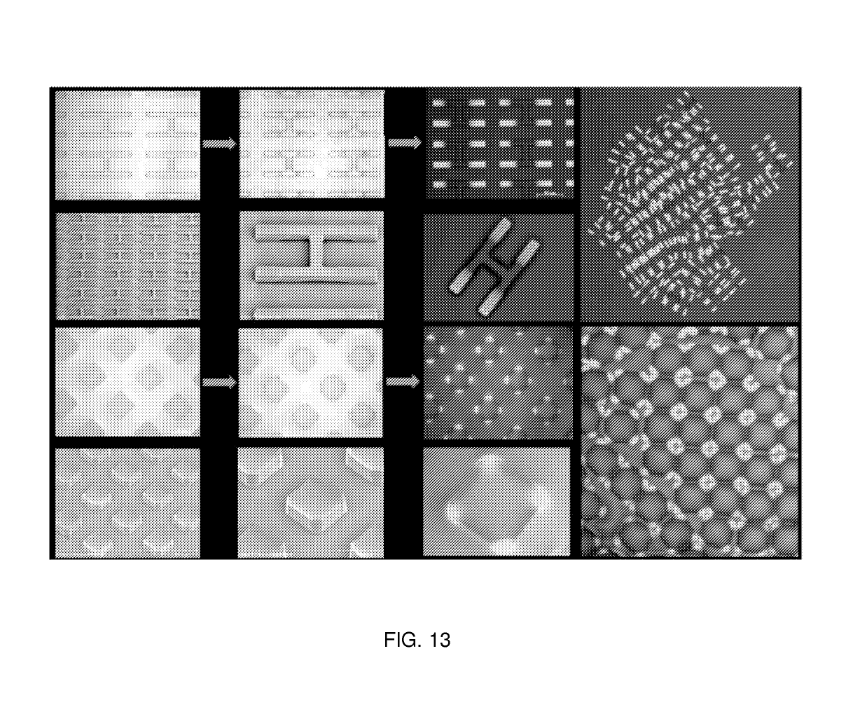

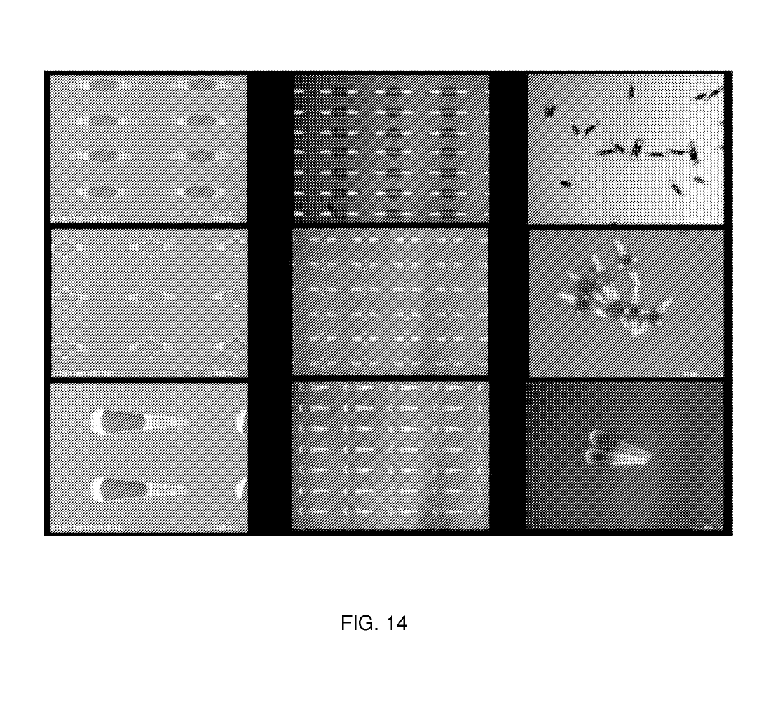

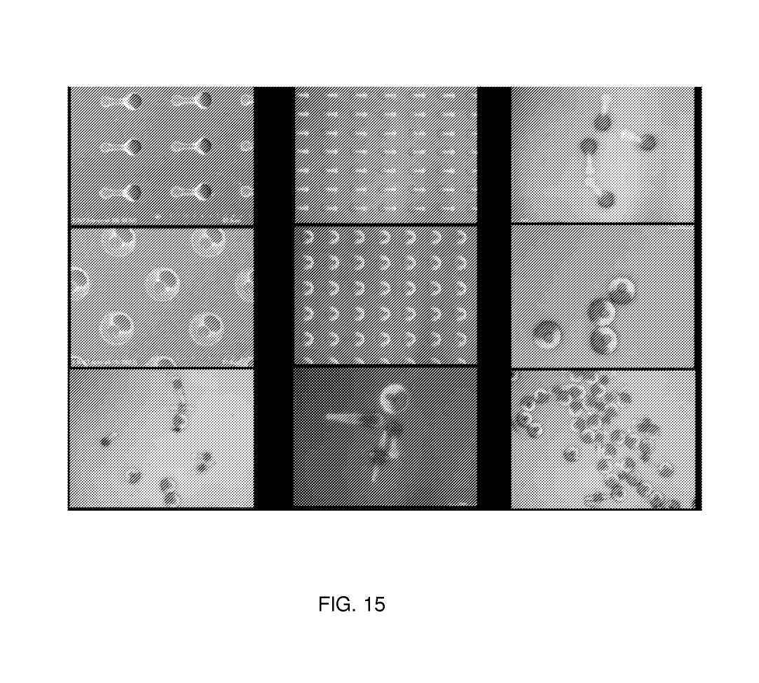

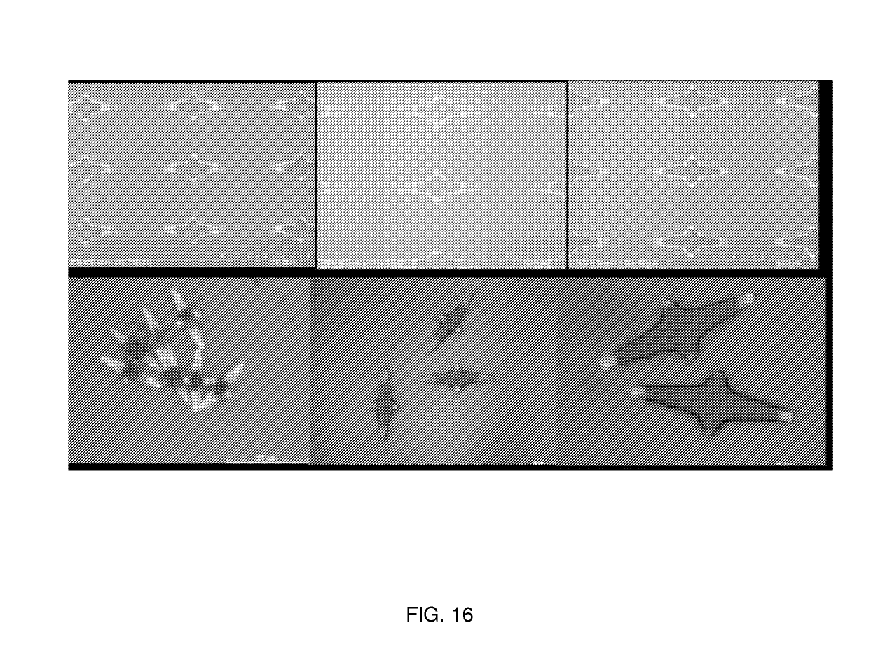

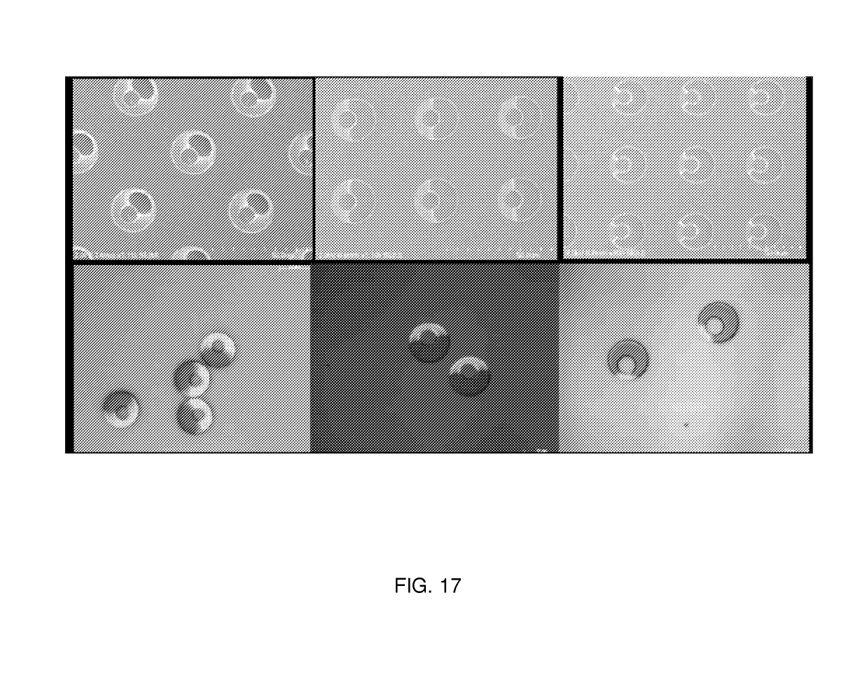

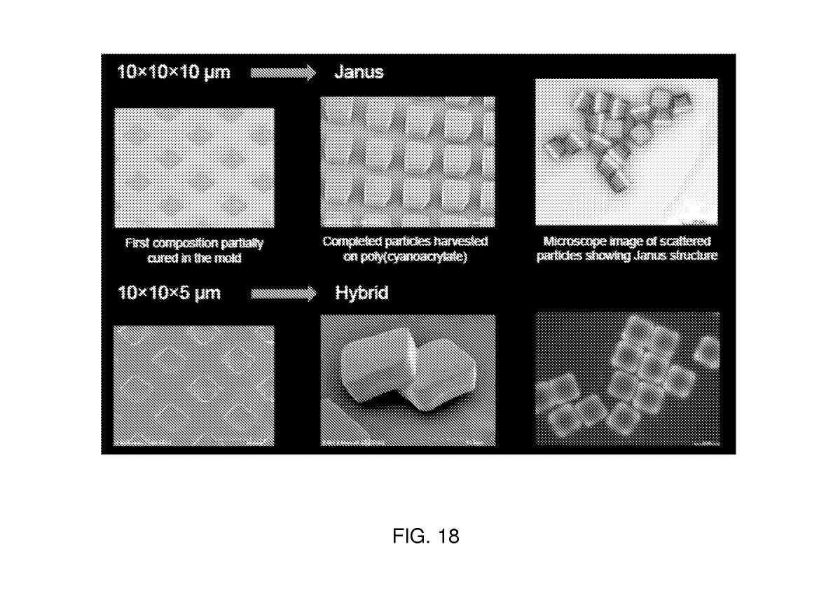

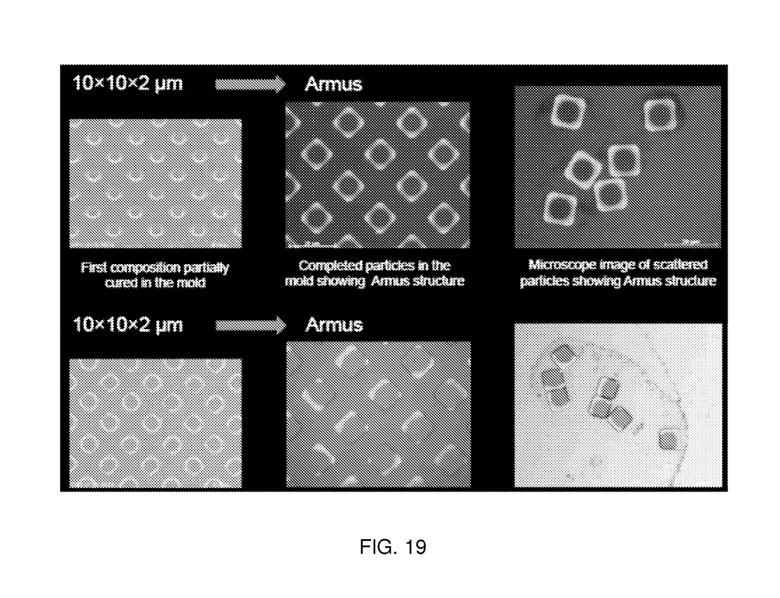

Advances in the field of nanotechnology, especially as it pertains to the design of nanometer- and micron-sized particles, have allowed for the fabrication of particles with sophisticated moieties, such as delicate cargo and surface-bound targeting ligands. However, in general, the distinct chemical species that compose a particle isotropically distribute in the particle to form either chemically or disordered alloys or core-shell layered structures. Controlling the distribution of matter in the particles allows for an extra parameter in the design process beyond the fundamental size and shape considerations, especially when the overall size and shape of the particle is controlled. It is advantageous to fabricate anisotropically phase-separated multiphasic particles owing to the resulting unique attributes not possible in single component or isotropically distributed multicomponent particle, as shown in FIGS. 7-19. These attributes include the ability to simultaneously utilize the different functions incorporated into the particle such as mechanical, chemical, optical, biological, electrical, and magnetic properties as well as the ability to function as multiple component carriers for drug delivery. Also, distinct functional ligands can be anisotropically arranged on the surface of particles, thus leading to unique properties fitted for various material and life science applications. Such behavior affords opportunities to create revolutionary new materials through combinations of different functionalities.

As discussed briefly above, the bulk density of the particle may be influenced by the introduction of material anisotropy according to one embodiment of the present invention. Of particular relevance to engineered particles is the density mismatch created within a single particle by the combination of two or more diverse compositions incorporated in a single particle in JANUS or ARMUS particles, the particles and methods of fabrication of which according to exemplary embodiments are shown and described in FIGS. 5 and 7-19 and U.S. patent Ser. No. 12/439,281 filed Sep. 30, 2009 entitled Nanoparticles Having Functional Additives for Self and Directed Assembly and Methods of Fabricating Same, which is incorporated by reference herein in its entirety. It follows that by appropriate selection of matrices, it is possible to create an asymmetrically-loaded particle from a symmetrical shape, thereby dramatically modifying its bulk density distribution and in turn, its aerodynamic performance. The density mismatch may be distinct with a well defined boundary between compositions within the same particle, or it may be graduated over the overall volume.

This principle may be further extended to the incorporation of nanoparticles of significantly higher densities (e.g., gold, silver or iron oxide nanoparticles) within the bulk matrix for the primary purpose of modulating overall density in addition to any diagnostic or therapeutic advantages afforded by such nanoparticles. The spatial location of these inclusions can be selectively sequestered in desired locations within the overall aerosol matrix. Thus, it is possible to create directionally-aligned particles predisposed to a particular mode of flight (e.g., tumbling or autorotation) or to introduce an additional mode to a shape previously predisposed to a single mode.

From a therapeutic perspective, the inclusion of two or more therapeutics in a single particle is particularly valuable in treating multi-drug resistant diseases or for co-delivery of diagnostic and therapeutic agents using multi-modal particles. A density mismatch or bulk anisotropy may be created by appropriate selection of therapeutics and excipients of appropriate densities (e.g., proteins and sugars).

This principle can also be used to selectively create porosity in a desired location (e.g., the radial arms or the core of an in-plane autorotating shape) or surface (e.g., the leading or trailing edges of an aerofoil) while leaving the remaining particle to be uniformly solid. The deposition pattern of these anisotropic or density-mismatched composites will be dependent on a combination of their shape, size as well as material anisotropy, but are likely to be distinct from their isotropic counterparts. Because of the complexity of their design, it may be necessary to predict the in vivo deposition patterns of such composite aerosols using CFD models.

Many modifications and other embodiments of the invention will come to mind to one skilled in the art to which this invention pertains having the benefit of the teachings presented in the foregoing description; and it will be apparent to those skilled in the art that variations and modifications of the present invention can be made without departing from the scope or spirit of the invention. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

The following examples are presented by way of illustration, not by way of limitation.

### EXPERIMENTAL

Using PRINT®, a class of “shaped” aerosols has been designed to include optimally engineered aerodynamic characteristics. This technique facilitates precise control over size, shape, and matrix properties, thereby allowing the ability to produce a wide range of clinically-relevant, therapeutic aerosols for targeted deposition in the respiratory tract.

Exemplary goals of the present invention include:
a. Develop PRINT® to fabricate engineered aerosols
b. Evaluate aerosolization characteristics
c. Demonstrate utility of PRINT® for aerosols
d. Evaluate various cargoes for delivery

The following examples provide proof of research and development in support of these exemplary goals.

### Results:

### Goal A: Develop PRINT® to Fabricate Engineered Aerosols

### Example 1

### Design of Novel Shapes for Engineered Aerosols

The uniqueness of shaped aerosols as related to PRINT® is its ability to adapt naturally occurring shapes as well as to design novel artificial (or engineered) shapes in order to facilitate enhanced and potentially tunable flight characteristics. This is a key distinction of PRINT® aerosols over that of the standard spherical shapes approximated by most commercially available aerosols. According to one embodiment, design parameters which influence aerodynamic properties include shapes that are:
i. non-spherical;
ii. symmetrical and promote autorotation about a central axis;
iii. asymmetrical and promote tumbling because of an unbalanced center of gravity (CG);
iv. capable of potentially generating lift by inducing leading edge vortices in addition to autorotation or tumbling;
v. fenestrated or that include cavities creating unbalanced CGs and thereby inducing autorotation, tumbling, and/or leading-edge vortices; and/or
vi. promote modulation of matrix anisotropy by preferentially redistributing mass in specific directions, facilitating the creating of JANUS-like particles.

A family of biomimetic shapes inspired by nature was designed to induce autorotation about a central axis and/or off-axis tumbling in an airstream (see e.g., FIGS. 3A-N discussed above). These are based on the hypothesis that autorotation and tumbling are likely to increase the time of flight of aerosols in pulmonary airways before impaction or deposition, thereby increasing the probability that these particles deposit deeper in the lung despite their relatively large size. Additionally, autorotation may be coupled to flow streamlines and may be influenced by secondary flows at airway bifurcations (in normal lungs) and obstructed airways (in case of COPD and asthma, for instance). It is expected that this would lead to anatomically differential deposition and to target specific airway generations and regions of the respiratory tract.

As discussed above, a series of ball-and-stick configurations were designed to promote autorotation and off-axis tumbling (see e.g., FIGS. 3F, J, M and N). Asymmetrical particles (see e.g., FIGS. 3J, M and N) are designed such that their CG is deliberately shifted away from at least one axis of symmetry to contrast with that of perfect spheres. Symmetrical tripod particles, shaped like helicopter propeller blades (see e.g., FIG. 3F), were inspired by maple seeds (samaras) that are known to disperse over long distance because of their ability to autorotate and generate leading edge vortices.²²

As also discussed above, the configuration shown in FIGS. 3C, 3D, 3E, 3K, and 3L are generally referred to as a “Lorenz” shape. This shape is modeled to induce two modes of flight: autorotation when solid (e.g., FIGS. 3C and K) and off-axis tumbling when an asymmetrical hole (fenestration) is introduced to shift its CG (see e.g., FIGS. 3D, E and L). This concept was also applied to an ellipsoid-like shape (see e.g., FIGS. 3A, B, G and H) that mimics pine seeds which are also known to be dispersed over long distances by wind.

Each of these shapes was approximately normalized to have a constant volume equivalent to that of a sphere with an ideal MMAD of 3 μm. The introduction of fenestrations in a given shape decreased its geometric volume, thereby decreasing the volume of the equivalent sphere and its potential MMAD. However, the aerodynamic properties of these aerosols are expected to be comparable to that of particles with MMADs in the 3-5 μm range, which is recommended for deep lung deposition.

### Example 2

### Microfabricated Templates for Engineered Aerosols

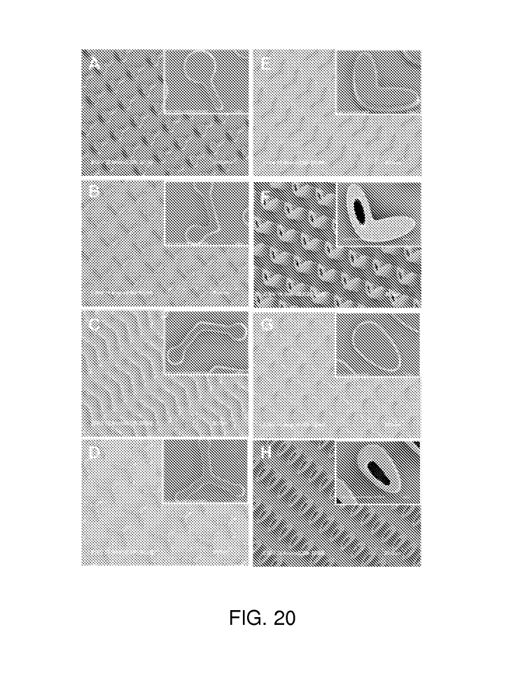

Microfabricated templates processed using traditional lithography techniques form the basis of shaped PRINT@ aerosols. Master templates for solid shapes were fabricated by exposing SU-8 negative resist (Microchem Corp, Newton, Mass.) to a 365 nm photolithography process on an I-line stepper. High aspect ratio features with fenestrations were resolved using a deep UV (193 nm) scanner (ASML, The Netherlands) on NFR 90 negative resist (JSR Micro Inc, Sunnyvale, Calif.). FIG. 20 illustrates SEM images of microfabricated templates for PRINT@ aerosols, wherein: (A) Lollipop; (B) L-Dumbbell; (C) V-Boomerang; (D) Helicopter; (E) Solid Lorenz; (F) Fenestrated Lorenz; (G) Solid Ellipsoid; (H) Fenestrated Ellipsoid.

Rolls of thin molds were then produced from these master templates using a proprietary technique developed by Liquidia Technologies (RTP Durham, N.C.). These molds allow for the roll-to-roll production of shaped aerosols. Furthermore, the same molds can be used to produce aerosols from a wide variety of compositions, as is described in Example 7, thereby demonstrating the versatility of the PRINT@ process in fabricating aerosols targeting various therapeutic applications.

### Example 3

### Fabrication of Engineered Aerosols

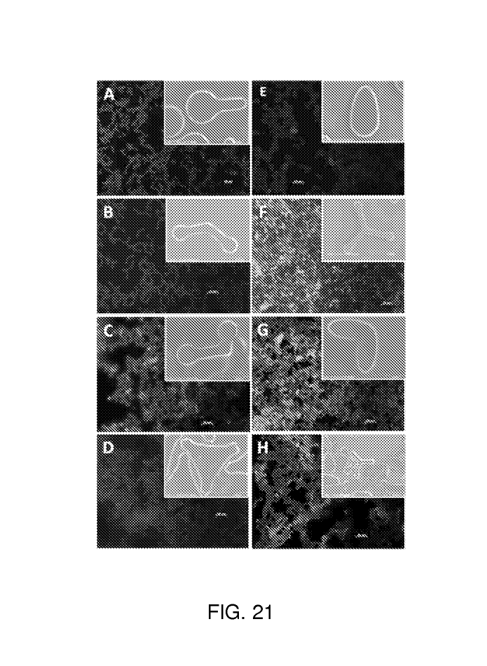

The PRINT@ process enables the fabrication of micron-sized aerosols. To demonstrate proof-of-concept, 7 different shapes were fabricated from a photocurable PEG hydrogel matrix as shown in FIG. 21. FIG. 21 shows Optical images (A-F) (100×) and SEM (inserts) images (2500×) of shaped PRINT@ aerosols, wherein: (A) Lollipops; (B) V-Boomerangs; (C) L-Dumbells; (D) Pollen; (E) Ellipsoids; (F) Helicopters; (G) Lorenz; (H) Mixed.

While the method of filling and photocuring this monomer in the molds has been previously demonstrated, a novel method of harvesting these aerosols to a PVOH sacrificial harvest layer (under specific temperature and pressure conditions) was developed. Furthermore, the incorporation of fluorescent dye cargo in these particles demonstrates the ability to use the particles as delivery vehicles for other diagnostic and therapeutic agents.

### Goal B: Evaluate Aerosol Characteristics

### Example 4

### Characterization of Engineered Aerosols

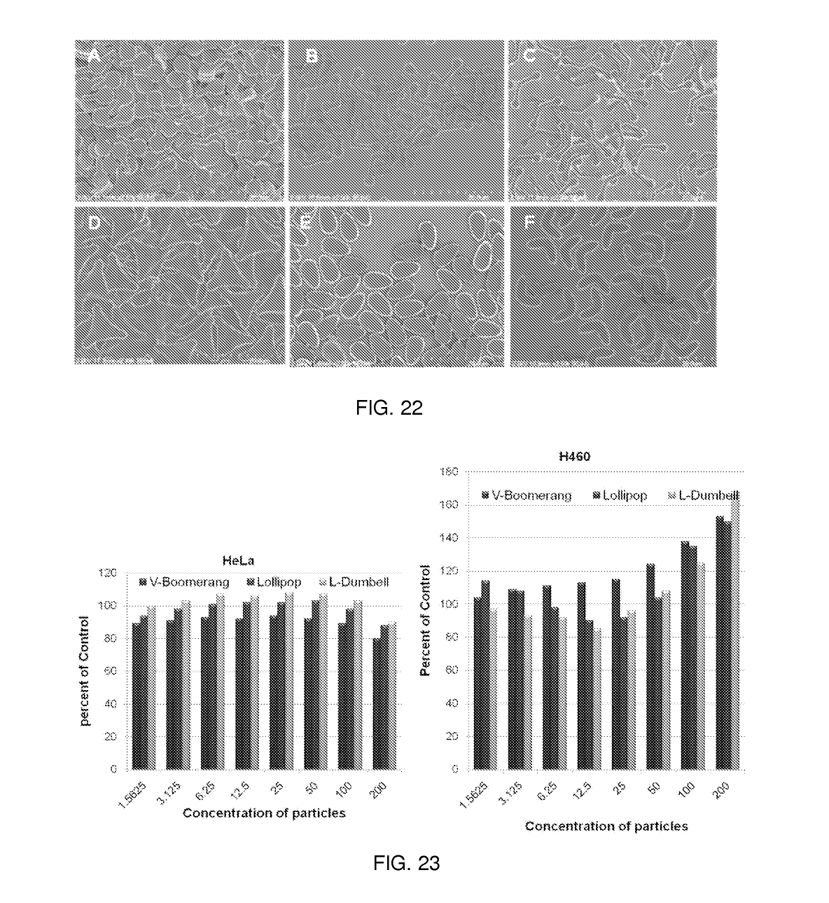

Physical characterization of aerosols is routinely done using optical and electron microscopy. As shown by the SEM images in FIG. 22, the aerosols are non-aggregated, distinct particles having well-defined edges. FIG. 22 depicts SEM images (2500×) of various shaped aerosols, wherein: (A) Lollipops; (B) L-Dumbells; (C) V-Boomerangs; (D) Pollen; (E) Ellipsoids; (F) Lorenz. Distinct, non-aggregated particles with well-defined and highly reproducible morphologies are shown. In keeping with the processing advantages of PRINT®, the aerosols are able to reproduce the exact morphology of the original microfabricated templates with a high level of fidelity.

Preliminary aerodynamic characterization has been performed using the aerodynamic particle sizer (APS). This light scattering technique quantifies key aerosol characteristics such as the Mass Mean Aerodynamic Diameter (MMAD) and the Geometric Standard Deviation (GSD) for each dry powder aerosol. Representative results from initial tests (Table 1) suggest a couple of key characteristics for PRINT® engineered aerosols. Firstly, the low GSD values as compared to most commercially available aerosols demonstrate the ability of the PRINT® process in fabricating non-aggregating aerosols with tight size distributions. Secondly, for the same shape, it is possible to produce aerosols with dramatically different and potentially scalable MMADs by scaling the template size appropriately, as demonstrated by the data for the 3 μm and 6 μm donuts. Finally, for aerosols with an equivalent overall design volume, there is still a distinct difference in MMADs based on their unique shapes, as shown by the differences in the values for the tripod helicopters and ellipsoids. This preliminary data demonstrates that it is possible to manipulate MMADs and thereby modulate pulmonary deposition profiles on the basis of tunable shapes and sizes for engineered aerosols.

TABLE 1
Summary of APS data for
representative PRINT ® aerosols.

| Shape | MMAD* (μm) | GSD** |
|---|---|---|
| 3 μm Donut | 2.23 | 1.6 |
| 6 μm Donut | 4.89 | 1.52 |
| Helicopter | 1.95 | 1.46 |
| Ellipsoid | 1.58 | 1.49 |

*Mass Mean Aerodynamic Diameter;
**Geometric Standard Deviation

### Example 5

### In Vitro Characterization of Cytotoxicity and Uptake

In preparation for in vivo deposition of the engineered aerosols, the PEG-based microparticles were tested for cytotoxicity in two different cells lines using an MTA assay. Following a 72-hour incubation, little to no cytotoxicity was observed (see FIG. 23). In particular, FIG. 23 illustrates cytotoxicity data for 72 hour incubation of PEG particles from the Ball-&-Stick family of shapes (e.g., Lollipop, V-Boomerang, and L-dumbbell). PVOH-harvested particles showed no detectable cytotoxicity with both HeLa and H460 cell lines across all 3 shapes. This assay is now built into the characterization of these aerosols.

### Example 6

### In Silico Characterization of Aerodynamic Performance

Using custom-built Computational Fluid Dynamics (CFD) modeling software, preliminary calculations have been performed to evaluate the settling of shaped aerosols under zero flow conditions, solely under the effect of gravity. This is a preliminary test to predict the aerodynamic behavior of these aerosols under realistic low Reynold's number and secondary flow conditions in the lungs. The settling time is computed by modeling the terminal velocity, i.e., the average (steady state) velocity of the particle at a terminal distance of 100 mm of free fall under gravity in air.

As shown in Table 2, settling times for individual shapes vary significantly with changes in overall shape. Shaped aerosols settle between 27-59% slower than equivalent spheres of comparable volume. Furthermore, the difference between settling times for same shape (ellipsoid) with and without fenestrations is ˜16%. This preliminary data suggests that shapes inducing autorotation or asymmetrical tumbling produce significant differences in flight characteristics.

TABLE 2
Settling Time Calculations for Shaped Aerosols

| | | Terminal | |
|---|---|---|---|
| | Simulation | Velocity* | Settling |
| Shape | Volume (μm³) | (μm/s) | Time (s) |
| Lollipop | 22.79 | 407.02 | 245.66 |
| Eqv. Sphere 1 | 22.80 | 596.31 | 167.87 |
| Lorenz | 33.5 | 509.71 | 196.19 |
| Ellipsoid | 32.81 | 467.11 | 214.09 |
| Eqv. Sphere 2 | 32.81 | 741.55 | 134.85 |
| Fenestrated | 27.83 | 543.49 | 184.00 |
| Ellipsoid | | | |
| Eqv. Sphere 3 | 27.83 | 690.00 | 144.86 |

*Terminal Velocity = Avg. (steady state) velocity in free fall
Eqv. Spheres 1, 2 and 3 = Volume-matched controls for Lollipop, Ellipsoid and fenestrated Ellipsoid respectively.

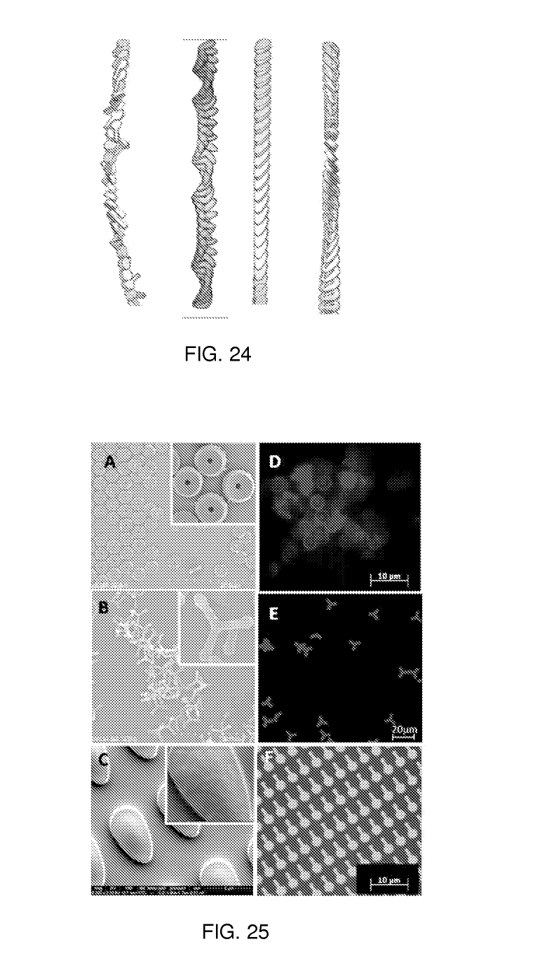

Furthermore, visualization of the settling profiles of these aerosols also show distinct differences in flight paths based on their shapes, as shown in FIG. 24, wherein the shapes include (from left to right) lollipop, Lorenz, ellipsoid, and fenestrated ellipsoid. Asymmetric lollipops demonstrate end-over-end tumbling as expected due to their off-axis CGs, whereas the Lorenz shape is mostly prone to autorotation as shown in FIG. 24. Solid ellipsoids show negligible rotation and have a relatively stable trajectory. In sharp contrast, fenestrated ellipsoids show a combination of both tumbling as well as autorotation.

Finally, as illustrated in FIG. 24, the autorotation of the Lorenz is not uniformly centered on a longitudinal axis or streamline. Rather, these aerosols trace a spiral about their central streamline, providing preliminary evidence for rifling in an airway. Based on these results, the choice of a symmetrical or asymmetrical design and the magnitude of the offset of the CG from the central axis may determine the radius of the spiral traced and thereby the extent of rifling. It is also believed that symmetrical autorotating particles are likely to adhere to flow streamlines and produce deep lung deposition, whereas asymmetrical autorotating (rifling) particles will likely impact earlier on the walls of airways that are smaller than the diameter of their defining spiral. These visualizations correlate well to the settling time calculations tabulated above, and provide additional support to the hypothesis of modulating aerosol aerodynamic properties. Thus, by designing particles of appropriate shapes and sizes, it is possible to influence the final pulmonary deposition pattern and, therefore, the diagnostic and therapeutic outcome of these aerosols.

### Goal C-D: Demonstrate Utility of PRINT@ for Aerosols and Evaluate Various Cargoes for Delivery

### Example 7

### Demonstrating Flexibility of the PRINT@ Platform for Various Matrices and Cargoes

One of the key strengths of the PRINT® process is its versatility in fabricating particles out of various compositions using the same mold. In the context of engineered aerosols, matrix flexibility is of great value in creating a wide variety of therapeutics with tunable aerodynamic properties. This is particularly true of therapies for multi-drug resistant pulmonary diseases like tuberculosis and lung cancer. Only recently has current aerosol technology progressed to fabricating aerosols capable of co-encapsulating two (or rarely three) therapeutics in the same vehicle. However, to the best of our knowledge, no other platform is capable of providing the flexibility afforded by PRINT® in encompassing as diverse a range of compositions as “neat” small molecule drugs to biological therapeutics. FIG. 25 shows aerosols made from some of these matrices as proof-of-concept, whereas Table 3 below lists the various matrices that have been used to test shaped aerosols to date. In particular, FIG. 25 illustrates SEM (A-C) and optical (D-F) images of PRINT® aerosols made of various matrices, wherein: (A) Lactose-BSA 3 mm Donuts; (B) Lactose-BSA Helicopters; (C) PLGA Ellipsoids; (D) Alexa-688 labeled Lactose-BSA Donuts; (E) Rhodamine-B labeled PEG Helicopters; (F) Fluorescein o-acrylate labeled PEG-HEA lollipops on a PVOH transfer sheet.

TABLE 3
List of various matrices tested for shaped aerosols

| Matrix | | Aerosol | |
|---|---|---|---|
| components | Imaging Agents | Shapes | Application |
| Lactose, BSA, | BSA-Alexa688 | Donuts, | Biologics, |
| Leucine, | conjugate | Pollen, | Protein |
| Glycerol | | Helicopters | therapeutics |
| PEG₇₀₀Diacrylate, | Fluorescein | Donuts, | Imaging of in |
| HEA^{#}, | o-acrylate, | Ellipsoids, | vivo deposition |
| AEM*, DEAP** | Rhodamine-B | Helicopters, | profiles |
| | | Lollipops | |
| PLGA, | N/A | Ellipsoids | Controlled |
| Ethylene | | | release, |
| Glycol | | | modulating |
| | | | matrix density |

^{#}Hydroxyethyl acrylate;
*Aminoethyl Methacrylate;
**2,2-Diethoxy acetophenone

### Example 8

### Modulating Matrix Anisotropy for PRINT® Engineered Aerosols

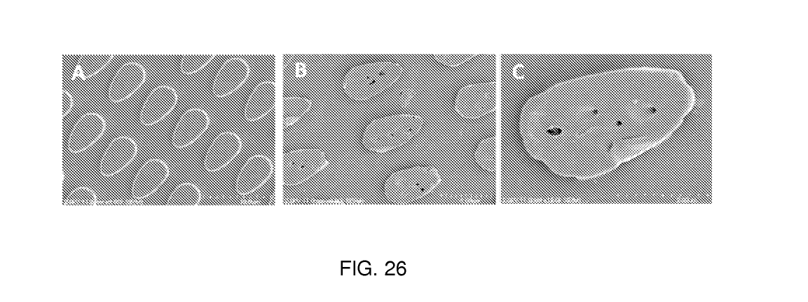

In addition to the flexibility of aerosol compositions as elucidated in Example 7, PRINT® also allows the modulation of bulk and surface properties of the aerosol matrix in order to influence its aerodynamic properties. One of the key parameters influencing the aerosol MMAD is the bulk density of its matrix. In fact, decreasing the aerosol matrix density by increasing its porosity allows relatively large particles (MMAD>10 μm) to behave like small solid particles (MMAD 3□μm) and deposit deep into the respiratory tract.²³ Preliminary experiments to fabricate porous particles out of a biodegradable PLGA matrix have been successful. While physical and aerodynamic characterization of these particles is in progress, the SEM images in FIG. 26 demonstrate the ability of PRINT® to influence a key material parameter (matrix density), thereby modulating the aerosols aerodynamic performance. In this regard, FIG. 26 illustrates porous PLGA particles with 20 wt % poly (vinyl pyrrolidine) as porogen (A) before and (B-C) after soaking in de-ionized water for 4 hours. Based on the aforementioned results, it may be possible to systematically influence pulmonary deposition from large airways to deep lungs simply by varying the matrix porosity from a low to high value for an aerosol of the same shape, size and composition.

### REFERENCES

1. “Direct Fabrication and Harvesting of Monodisperse, Shape Specific Nano-Biomaterials”; Rolland, J. P.; Maynor, B. W.; Euliss, L. E.; Exner, A. E.; Denison, G. M.; DeSimone, J. M J. Am. Chem. Soc. 2005, 127, 10096.
2. “The Pursuit of a Scalable Nano-fabrication Platform for Use in Material and Life Science Applications”; Gratton; Williams; Napier; Pohlhaus; Zhou; Wiles; Maynor; Shen; Olafsen; Samulski; DeSimone Accounts of Chemical Research 2008, 41, 1685.
3. “Imparting Size, Shape, and Composition Control of Materials for Nanomedicine”; Eulis, L.; DuPont, J.; DeSimone, J. M. Chem. Soc. Rev. 2006, 35, 1095.
4. “The Effect of Particle Design on Cellular Internalization Pathways”; Gratton, S. E. A.; Ropp, P. A.; Pohlhaus, P. D.; Luft, J. C.; Madden, V. J.; Napier, M. E.; DeSimone, J. M. Proceedings of the National Academy of Sciences 2008, 105(33), 11613.
5. “Reductively Labile PRINT Nanoparticles for the Delivery of Doxorubicin to HeLa Cells”; Petros, R. A.; Ropp, P. A.; DeSimone, J. M.; J. Am. Chem. Soc. 2008, 130, 5008.
6. “Electrically-Driven Alignment and Crystallization of Unique Anisotropic Polymer Particles”; Herlihy, K. P.; Nunes; J. DeSimone, J. M.; Langmuir 2008, 24, 8421.
7. “Shape-specific, Mono-disperse Nano-molding of Protein Particles”; Kelly, J. Y.; DeSimone, J. M. J. Am. Chem. Soc. 2008, 130, 5438.
8. “Soft Lithography”; Y. Xia, G. M. Whitesides, Angew. Chem. 1998, 110, 568.
9. “Contact Angle Analysis, Surface Dynamics, and Biofouling Characteristics of Cross-linkable, Random Perfluoropolyether-based Graft Ter-polymers”; Yarbrough; Rolland, J. P.; DeSimone, J. M.; Callow, M. E.; Finlay, J. A.; Callow, J. A. Macromolecules 2006, 39, 2521
10. “Optically Transparent, Amphiphilic Networks Based on Blends of Perfluoropolyethers and Poly(ethylene glycol)”; DeSimone et. al. Am. Chem. Soc. 2008, 130, 14244
11. “Superhydrophobic Behavior of a Perfluoropolyether Lotus-Leaf-Like Topography”; Zhang, L.; Zhou, Z.; Cheng, B.; DeSimone, J. M.; Samulski, E. T. Langmuir 2006, 22, 8576
12. “Supramolecular Nano-mimetics: Replication of Micelles, Viruses and Other Naturally-Occurring Nanoscale Objects”; Maynor; LaRue; Hu; Rolland; Pandya; Fu; Liu; Spontak; Sheiko; Samulski; Samulski; DeSimone Small 2007 3(5), 845
13. “Weak Surface Anchoring Energy of 4-Cyano-4′-pentyl-1,1′-biphenyl on Perfluoropolyether Langmuir-Blodgett Films”; Russell-Tanner, J. M.; Takayama, S.; Sugimura, A.; DeSimone, J. M.; Samulski, E. T. J. Chem. Phys. 2007, 126 (24): Art. No. 244706.
14. “Delivery of biological agents by aerosols”, Edwards D A. Aiche Journal. 2002, 48, 2-6.
15. “Deposition of Particles in the Human Respiratory-Tract” Heyder J, Gebhart J, Rudolf G, Schiller C F, Stahlhofen W. Journal of Aerosol Science 1986, 17, 811.
16. “Physical characterization of component particles included in dry powder inhalers. I. Strategy review and static characteristics”; Hickey et al. J. of Pharmaceutical Sci 2007, 96, 1282.
17. “Physical characterization of component particles included in dry powder inhalers. II. Dynamic characteristics.”; Hickey et al. Journal of Pharmaceutical Sci 2007, 96, 1302.
18. “Templated Open Flocs of Nanorods for Enhanced Pulmonary Delivery with Pressurized Metered Dose Inhalers” Engstrom et. al. Pharmaceutical Research. 2009, 26, 101.
19. “Leading Edge Vortices Elevate Lift of Autorotating Plant Seeds”; Lentink, et. al. Science 2009, 324, 1438.
20. “Large Porous Particles for Pulmonary Delivery”; Edwards, et. al. Science 1997, 276, 1868.
21. “Drug Delivery to the Central Nervous System: A Review”; Misra, A.; Shahiwala, A. J. Pharmceut Sci. 2003, 6, 252.
22. Letnik D, Dickson W B, van Leeuwen J L, Dickinson M H, “Leading-edge vortices elevate lift of autorotating seeds.” Science 2009 324:1438-1440.
23. Edwards D A, Hanes J, Caponetti G, Hrkach J, BenJebria A, Eskew M L, Mintzes J, Deaver D, Lotan N, Langer R ‘Large porous particles for pulmonary drug delivery’ SCIENCE 276 1997 1868-1871.

1. A collection of engineered particles, wherein each particle comprises:
a fabricated nanoparticle body member being non-spherical and configured to provide tumbling when entrained in an airstream to thereby increase settling time of the fabricated nanoparticle body member, wherein the nanoparticle body member has a three dimensional shape consisting of;
a longest dimension defining a longitudinal axis of the three dimensional shape; a uniform thickness; and,
an off-center fenestration wherein the off-center fenestration provides the particle with one dimension of symmetry and a center of mass offset from the longitudinal axis.

2. The collection of engineered particles of claim 1, wherein the fabricated nanoparticle body member is configured to settle between about 27-59% slower than an equivalent sphere of comparable volume.

3.-5. (canceled)

6. The collection of engineered particles of claim 1,
wherein the fenestration is non-circular.

7. The collection of engineered particles of claim 1, wherein the fenestration is offset with respect to the longitudinal axis of the fabricated nanoparticle body member.

8. The collection of engineered particles of claim 1, wherein the fabricated nanoparticle body member has a non-uniform density distribution.

9. The collection of engineered particles of claim 8, wherein the fabricated nanoparticle body member comprises a plurality of phase-separated materials.

10. The collection of engineered particles of claim 8, wherein the fabricated nanoparticle body member is porous.

11. The collection of engineered articles of claim 8, wherein the fabricated nanoparticle body member comprises a plurality of compositions having a different density from one another.

12. (canceled)

13. The collection of engineered particles of claim 1, wherein the fabricated nanoparticle body member is configured to carry a cargo therewith for delivering the cargo to a delivery site.

14. The collection of engineered particles of claim 13, wherein the cargo is selected from the group consisting of: a therapeutic agent, a pharmaceutical agent, a tag, a magnetic material, a paramagnetic material, a superparamagnetic material, a sensing agent, a signaling agent, a taggant, an imaging agent, a charged species, a biologic agent, a diagnostic agent, a drug, and combinations thereof.

15. The engineering particle of claim 1, wherein the fabricated nanoparticle body member is configured to provide autorotation or lift.

16.-20. (canceled)

21. The engineering particle of claim 1, wherein the fabricated nanoparticle body member is configured to provide lift through creation of a leading edge vortex.

22. (canceled)

23. The collection of engineered particles of claim 1, wherein the fabricated nanoparticle body member has a shape of a fenestrated ellipsoid.

24. The collection of engineered particles of claim 1, wherein the center of mass comprises a center of gravity.

25. A method of delivering an engineered aerosol particle, the method comprising:
providing in aerosol form a plurality of fabricated nanoparticle body members being non-spherical and configured to provide at least one of auto-rotation, tumbling, or lift when entrained in an airstream; and
releasing the fabricated nanoparticle body members into an airstream.

26. (canceled)

27. A method for delivering at least one therapeutic agent to a subject, said method comprising administering a plurality of engineered nanoparticles comprising said therapeutic agent to said subject via pulmonary inhalation or via intranasal administration to achieve delivery to the central nervous system, wherein at least one of said engineered nanoparticles comprises a microfabricated nanoparticle body member being non-spherical and configured to provide at least one of auto-rotation, tumbling, or lift when entrained in an airstream.

28.-29. (canceled)
